(19) United States
(12) Patent Application Publication
(10) Pub. No.: US 2024/0325264 A1
(43) Pub. Date: Oct. 3, 2024

(54) COMPOSITIONS COMPRISING BIOACTIVE GLASS AND AMINO ACID

(71) Applicant: CORNING INCORPORATED, CORNING, NY (US)

(72) Inventors: Qiang Fu, Painted Post, NY (US); Nicholas Michael Stone-Weiss, Corning, NY (US); Joanna E. Warzyszynska, Painted Post, NY (US)

(21) Appl. No.: 18/613,904

(22) Filed: Mar. 22, 2024

Related U.S. Application Data
(60) Provisional application No. 63/608,465, filed on Dec. 11, 2023.
(60) Provisional application No. 63/455,484, filed on Mar. 29, 2023.

(51) Int. Cl. A61K 8/27; A61K 8/24; A61Q 11/00; A61Q 19/08

(52) U.S. Cl. CPC A61K 8/27; A61K 8/24; A61Q 11/00; A61Q 19/08; A61K 2800/5922; A61K 2800/60; A61K 2800/80

(57) ABSTRACT

A composition comprising an amino acid and a bioactive glass, in which the bioactive glass comprises 15-55 wt. % SiO₂, 20-60 wt. % CaO, 0.1-15 wt. % ZrO₂, and 5-30 wt. % P₂O₅, in which, when the composition is subjected to artificial saliva at 37° C., a bioactive crystalline phase is formed within 7 days. In some aspects, the bioactive crystalline phase is apatite, and the composition is a dentifrice or skin care composition. Methods comprising making the composition, making the bioactive glass, and applying the composition to a tooth or the skin. In some aspects, applying the composition to a tooth remineralizes enamel of the tooth, treats caries of the tooth, and/or treats dentin hypersensitivity of the tooth, and applying the composition to skin treats or reduces the appearance of one or more skin defects, such as wrinkles and blemishes.

### CROSS-REFERENCE TO RELATED APPLICATIONS

This application claims the benefit of priority of U.S. Provisional Application 63/608,465, filed on Dec. 11, 2023, and U.S. Provisional Application 63/455,484, filed on Mar. 29, 2023, the content of each of which is relied upon and incorporated herein by reference in its entirety.

### FIELD

This disclosure generally relates to bioactive glasses, and more particularly to bioactive glasses for consumer, dental, and cosmetic applications.

### BACKGROUND

Bioactive glasses are a group of glass and/or glass ceramic materials that have shown biocompatibility or bioactivity, which has allowed them to be incorporated into human or animal physiology. Generally speaking, bioactive glasses may be able to bond with hard and soft tissues, thereby fostering growth of bone and cartilage cells. Moreover, bioactive glasses may also enable release of ions which activate expression of osteogenic genes and stimulate angiogenesis, as well as promote vascularization, wound healing, and cardiac, lung, nerve, gastrointestinal, urinary tract, and laryngeal tissue repair.

Some bioactive glasses, such as 45S5 Bioglass® sold by Haleon under the Sensodyne® brand featuring NovaMin® technology, are being investigated for their potential ability to mineralize teeth; however, the low chemical durability of these traditional bioactive glasses is problematic for compositions requiring prolonged shelf times in aqueous environments. For example, 45S5 Bioglass® requires development of a non-aqueous environment for glass particulates to be used in toothpaste applications. In addition, the speed at which 45S5 Bioglass® may mineralize teeth also is not ideal.

Thus, there is a need for improved bioactive glasses and compositions thereof.

### SUMMARY

In some aspects, disclosed is a composition comprising:
an amino acid; and
a bioactive glass, comprising:
   15-55 wt. % SiO₂;
   0.1-15 wt. % ZrO₂;
   20-60 wt. % CaO; and
   5-30 wt. % P₂O₅;
   wherein, when the composition is subjected to artificial saliva of Table 1A or 1B at 37° C., a bioactive crystalline phase is formed within 7 days.

In some aspects, the composition is a dentifrice composition or a skin care composition.

In some aspects, a dental strip or dental tray comprises the composition, optionally with additional components such as one or more of water, glycerin, a humectant, a fluoride ion source, a flavoring, a surfactant, a sweetener, a whitening agent, an abrasive, a thickening agent, a pH modifier, a preservative, or any combination thereof.

In some aspects, disclosed is a method for making the composition, comprising combining the amino acid, the bioactive glass, and optionally one or more of water, glycerin, a humectant, a fluoride ion source, a flavoring, a surfactant, a sweetener, a whitening agent, an abrasive, a thickening agent, a pH modifier, a preservative, or any combination thereof.

In some aspects, disclosed is a method comprising applying the composition to a bone (e.g., a tooth). In some aspects, the method remineralizes enamel of the tooth, treats caries of the tooth, treats dentin hypersensitivity of the tooth, or any combination thereof.

In some aspects, disclosed is a method comprising applying the composition to skin. In some aspects, the method treats or reduces the appearance of one or more skin defects, optionally wherein the one or more skin defects comprises wrinkles, blemishes, sagging skin, or any combination thereof.

In some aspects, disclosed is a method for making the bioactive glass, comprising:
combining each component of the bioactive glass to form a mixture,
heating the mixture to a temperature of 1500° C. or less to form a melted mixture, and
cooling the melted mixture to form a cooled mixture.

Additional features and advantages of the disclosure will be set forth in the detailed description which follows, and in part will be readily apparent to those skilled in the art from that description or recognized by practicing the aspects as described herein, including the detailed description which follows, the claims, as well as the appended drawings.

It is to be understood that both the foregoing general description and the following detailed description are merely exemplary and are intended to provide an overview or framework for understanding the nature and character of the disclosure and claims. The accompanying drawings are included to provide a further understanding of the disclosure and are incorporated into and constitute a part of this specification. The drawings illustrate various aspects of the disclosure and together with the description serve to explain the principles and operations of the various aspects.

### BRIEF DESCRIPTION OF THE DRAWINGS

The following detailed description can be further understood when read in conjunction with the following drawings. Whenever possible, the same reference numerals will be used throughout the drawings to refer to the same or like parts. It is to be understood that the figures are not drawn to scale and the size of each depicted component or the relative size of one component to another is not intended to be limiting.

FIG. 1 is a powder X-ray diffraction (XRD) spectrum illustrating hydroxyapatite formation within 1 day for an example bioactive glass upon immersion in an aqueous composition comprising artificial saliva and an amino acid (glycine).

FIG. 2 is a powder XRD spectrum illustrating hydroxyapatite and brushite formation within one day for an example bioactive glass upon immersion in an aqueous composition comprising artificial saliva and an amino acid (glutamic acid), followed by hydroxyapatite being the only phase within 4 and 7 days.

FIG. 3 is a powder XRD spectrum illustrating hydroxyapatite formation at 7 days for an example bioactive glass upon immersion in an aqueous composition comprising artificial saliva and an amino acid (arginine).

FIG. 4 is a powder XRD spectrum illustrating no hydroxyapatite formation within 7 days for a comparative glass composition (45S5 Bioglass®) upon immersion in an aqueous composition comprising artificial saliva and an amino acid (arginine).

FIG. 5 is a graph illustrating pH change over time upon separately immersing an example bioactive glass in an aqueous composition comprising artificial saliva and three different amino acids (glycine, glutamic acid, and arginine).

FIG. 6 is a graph illustrating pH change over time upon separately immersing an example bioactive glass and a comparative glass composition (45S5 Bioglass®) in an aqueous composition comprising artificial saliva and an amino acid (arginine).

FIG. 7 is a powder XRD spectrum illustrating no crystal phase formation within 7 days for a comparative glass composition (45S5 Bioglass®) upon immersion in artificial saliva.

FIG. 8 is a powder XRD spectrum illustrating apatite formation occurred within 4 days of soaking bioactive glass in artificial saliva without amino acid present, and significant additional formation of apatite did not occur between days 4 and 7.

FIGS. 9A, 9B, and 9C are scanning electron microscopy (SEM) images illustrating crystal growth on the surface of a bioactive glass disclosed herein after immersion in artificial saliva comprising glycine.

FIGS. 10A, 10B, and 10C are SEM images illustrating crystal growth on the surface of a bioactive glass disclosed herein after immersion in artificial saliva comprising glutamic acid.

FIGS. 11A, 11B, and 11C are SEM images illustrating crystal growth on the surface of a bioactive glass disclosed herein after immersion in artificial saliva comprising arginine.

FIGS. 12A, 12B, 12C, 12D, 12E, and 12F are SEM images illustrating the surface morphology, including presence or absence of crystal growth, of a human enamel disc after immersion in Table 5's artificial saliva composition for 4 days at 37° C. along with no other components (FIGS. 12A and 12B, which are different magnifications of the same sample), with 5 wt. % of Composition 1 of Table 3 (FIGS. 12C and 12D, which are different magnifications of the same sample), or with 5 wt. % CaCO₃ (FIGS. 12E and 12F, which are different magnifications of the same sample). None of these compositions contained amino acid.

FIGS. 13A, 13B, 13C, and 13D are SEM images illustrating surface morphology, including presence or absence of crystal growth, of a human enamel disc after immersion in Table 5's artificial saliva composition with 5 wt. % of Composition 1 of Table 3 and 8 wt. % of glycine at 37° C. for 1 day (FIGS. 13A and 13B, which are different magnifications of the same sample), or after immersion in Table 5's artificial saliva solution with 45S5 Bioglass® and 8 wt. % glycine (FIGS. 13C and 13D, which are different magnifications of the same sample) at 37° C. for 4 days.

FIGS. 14A, 14B, 14C, and 14D are SEM images with different magnifications of the same sample illustrating surface morphology and cross-sectional view of the remineralization of human enamel discs after immersion for 1 day at 37° C. in Table 5's artificial saliva composition with 5 wt. % of Composition 1 of Table 3 and 8 wt. % of glycine.

FIG. 15A is a powder XRD spectrum illustrating hydroxyapatite formation occurred within 4 hours of immersing 5 wt. % of Composition 1 bioactive glass in artificial saliva containing 25 wt. % glycine.

FIG. 15B is a powder XRD spectrum comparing apatite or calcite formation when immersing 5 wt. % Composition 1 bioactive glass, 5 wt. % 45S5 Bioglass®, or 5 wt. % calcium carbonate in artificial saliva containing 8 wt. % glycine.

FIG. 16 is a scatter plot illustrating weight loss from Composition 1 bioactive glass and 45S5 Bioglass® as a function of immersion time in DI water at 37° C.

FIGS. 17A-17B are SEM images of the surface morphology of a human enamel disc after treatment with a toothpaste that did not contain bioactive glass. FIG. 17B is a magnification of the boxed area of FIG. 17A.

FIGS. 18A-18B are SEM images of the surface morphology of a human enamel disc after treatment with a toothpaste containing bioactive glass. FIG. 18B is a magnification of the boxed area of FIG. 18A.

### DETAILED DESCRIPTION

In the following description, whenever a group is described as comprising at least one of a group of elements and combinations thereof, it is understood that the group may comprise, consist essentially of, or consist of any number of those elements recited, either individually or in combination with each other. Similarly, whenever a group is described as consisting of at least one of a group of elements or combinations thereof, it is understood that the group may consist of any number of those elements recited, either individually or in combination with each other.

Where a range of numerical values is recited herein, comprising upper and lower values, unless otherwise stated in specific circumstances, the range is intended to include the endpoints thereof, and all integers and fractions within the range. Further, when an amount, concentration, or other value or parameter is given as a range, one or more ranges, or a list of upper values and lower values, this is to be understood as specifically disclosing all ranges formed from any pair of any upper range limit or value and any lower range limit or value, regardless of whether such pairs are separately disclosed.

If the term “about” is used in describing a value or an end-point of a range, the disclosure should be understood to include the specific value or end-point referred to. As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. It is noted that the terms “substantially” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. Thus, for example, a glass that is “free” or “essentially free” or “substantially free” of Al₂O₃(or any other component) is one in which Al₂O₃(or any other component) is not actively added or batched into the glass, but may be present in very small amounts as a contaminant (e.g., 500, 400, 300, 200, or 100 ppm or less).

As used herein, a glass, glass-ceramic, or crystalline phase that is “bioactive” means that the glass, glass-ceramic, or crystalline phase is biologically compatible with bone, teeth, and/or tissue. By way of illustration, in some aspects, “bioactive” in this context refers to (1) the ability to form apatite (or other bioactive crystalline phases such as hydroxyapatite, fluorapatite, carbonated apatite, or any combination thereof) in a simulated body fluid, such as artificial saliva, according to ASTM F1538-03 (2017), and/or (2) the capability of binding with a desired biological material (e.g., bones, teeth, and/or tissue). Generally, there is also an absence of toxicity or other significant negative effects in a biological environment (e.g., bones, teeth, and/or tissue).

Herein, bioactive glasses are expressed in terms of wt. % amounts of particular components included therein on an oxide bases unless otherwise indicated. Any component having more than one oxidation state may be present in a bioactive glass in any oxidation state. However, concentrations of such component are expressed in terms of the oxide in which such component is at its lowest oxidation state unless otherwise indicated.

Oral diseases pose a major health burden worldwide, causing pain, discomfort, disfigurement, and even death. Commonly experienced issues include dental caries, erosive tooth wear affecting mechanical/aesthetic properties of the tooth, and hypersensitivity. Dental caries mediated by bacterial plaque acid production, and enamel acid erosion mediated by consumption of acidic beverages and foods result from the dissolution of enamel hydroxyapatite in acidic environments leading to mineral loss from the enamel layer. The dissolution of apatite crystals and the net loss of calcium, phosphate, and other ions from the tooth (i.e., demineralization) leads to dental caries formation and dentin hypersensitivity. Dentin hypersensitivity in particular can occur through erosion of the enamel, causing exposure of dentin tubules and leading to increased sensitivity to air and/or cold or hot temperatures in the mouth.

Current treatments and prophylactic measures for maintaining enamel include fluoride-based approaches, which lack an external source of calcium and phosphate ion provision for hydroxyapatite mineralization, and instead rely on salivary provision of these ions which may not be sufficient in all cases. Instead, caries and dentin hypersensitivity can be managed non-invasively through a remineralization process, in which calcium and phosphate ions are supplied from an external source to the tooth to promote crystal deposition into voids in demineralized enamel and at the dentin surface to occlude fluid flow causing hypersensitivity. Calcium phosphate phases in both crystalline form (e.g., brushite, β-tricalcium phosphate, octocalcium phosphate, hydroxyapatite, fluorapatite and enamel apatite) and amorphous form have been used in remineralization processes. However, due to their poor solubility in artificial and real saliva, crystalline calcium phosphates including brushite, β-tricalcium phosphate, octacalcium phosphate and apatite, cannot liberate calcium and phosphorus ions capable of diffusing into enamel subsurface lesions for efficient remineralization. In contrast, use of amorphous calcium phosphate (e.g., bioactive glass) in remineralization processes has shown promising results. Generally, bioactive glasses disclosed herein dissolve at a controlled rate in the mouth and convert to phases such as hydroxyapatite, fluorapatite, and other calcium phosphate phases that are compatible with tooth mineral.

45S5 Bioglass®, described in U.S. Pat. No. 4,234,972, is a glass known to convert to apatite; however, as shown in at least Examples 1 and 3, no detectable crystalline phases were observed for 45S5 Bioglass® after immersion for 7 days in a composition comprising artificial saliva both with (Example 1) and without (Example 3) amino acid. It is desirable to form an apatite phase from bioactive glass within a reasonable amount of time.

Treatments intended for enamel remineralization and/or treatment of hypersensitivity may be applied in many forms e.g. dentifrice, mouthwash, varnish and even chewing gum. In the last couple of decades, the use of in-home dental treatments utilizing strip- or tray-based delivery methods has gained popularity, particularly with respect to tooth whitening applications. Strip/tray-based dental products allow for directed delivery of an active formulation to the tooth surface and rely on the rapid action of their active ingredients. One such example is a sensitivity product from Procter and Gamble: Crest Sensistop™ Strips—a fast-acting oxalate-crystal based technology for physical occlusion of dentin tubules. This product, however, does not remineralize the enamel surface but instead relies on deposition of calcium oxalate crystals within dentin tubules.

Thus, there is a strong desire to develop new bioactive glasses and modes of delivery that take advantage of the remineralization process to prevent or repair tooth caries and/or dentin hypersensitivity.

Forming apatite in artificial saliva with the bioactive glasses disclosed herein is possible at the 4-day mark (see Example 1) when there is no amino acid present. However, it was found that by including amino acid, crystal phase formation occurs much more rapidly, within 1 day. This step change in the rate of formation of biologically compatible mineral phases represents a significant finding that can have monumental impact in the field of oral care, such as for the rapid treatment of tooth sensitivity or for dental enamel repair.

See also Examples 4-6 for an illustration of additional aspects comparing the bioactive glasses disclosed herein with a comparative 45S5 Bioglass® or CaCO₃.

In terms of tooth sensitivity, some previous attempts have also utilized CaCO₃. However, bioactive glasses, such as those disclosed herein, in some aspects show a key advantage since they display in some cases at least 10 times faster calcium release in aqueous environments compared to CaCO₃, while also providing dissolved phosphate. The disclosed bioactive glasses therefore provide two solution components, calcium and phosphate, for forming hydroxyapatite. Thus, in some aspects, the present approach uses a unique bioactive glass to mineralize hydroxyapatite, sometimes in combination with one or more amino acids to control the rate of mineralization (e.g., by increasing the rate of mineralization). In some aspects, the compositions disclosed herein are substantially free of, or do not contain, CaCO₃.

In some aspects, disclosed are bioactive glasses in combination with amino acid to rapidly form apatite in artificial saliva. In some aspects, the amino acids can be employed at various concentrations. In some aspects, the disclosed bioactive glasses display significantly improved mineralization behavior in comparison to 45S5 Bioglass®. In some aspects, the mineralization behavior represents a step-change in the rate of re-mineralization from the current technology in the oral care field, which may benefit multiple oral health applications.

In some aspects, the disclosed compositions rapidly mineralize crystalline phases on the surface of bioactive glasses in artificial saliva solutions. In some aspects, one or more amino acids (e.g., at various concentrations) in combination with a bioactive glass remineralizes tooth-compatible phases, such as hydroxyapatite or brushite, on the glass surface. In some aspects, the disclosed compositions rapidly mineralize crystalline phases on the surface of teeth in artificial saliva solutions. In some aspects, one or more amino acids (e.g., at various concentrations) in combination with a bioactive glass remineralizes tooth-compatible phases, such as hydroxyapatite or brushite, on the tooth surface. In some aspects, without wishing to be bound by theory, it is believed that the bioactive glass acts as the source of dissolved ions in the artificial saliva, while the amino acid may act to promote the nucleation and/or growth of crystalline phases. It is anticipated that the disclosed compositions will perform similarly in real saliva or other aqueous compositions.

In some aspects, the combination of bioactive glass and amino acid disclosed herein allows for rapid remineralization of tooth enamel surface in terms of improved remineralization rate and effectiveness with respect to other technologies such as 45S5 Bioglass® and arginine-calcium carbonate, which in turn allows for delivery of these ingredients in various configurations, including in a toothpaste, in a dental strip, and/or in dental tray. Additionally, in some aspects, improved water durability of the bioactive glasses disclosed herein relative to other commercially available bioactive glasses such as 45S5 Bioglass®, allows for use of an aqueous-based formulation (rather than glycerol-based formulation). Aqueous-based formulations are cheaper, representing a cost savings.

In some aspects, disclosed is a composition, comprising:
an amino acid; and
a bioactive glass, comprising:
   15-55 wt. % SiO₂;
   0.1-15 wt. % ZrO₂;
   20-60 wt. % CaO; and
   5-30 wt. % P₂O₅;
   based on total weight of bioactive glass;
wherein, when the composition is subjected to an artificial saliva of Table 1A or 1B at 37° C., a bioactive crystalline phase is formed within 7 days (or any of the other timeframes specified herein, such as within 1 day).

Bioactive glasses are a group of glass and/or glass ceramic materials that have shown biocompatibility or bioactivity, which has allowed them to be incorporated into human or animal physiology. In some aspects, disclosed is a bioactive glass comprising SiO₂, CaO, and P₂O₅. In some aspects, the bioactive glass further comprises ZrO₂ and/or F⁻. In some aspects, a composition containing an amino acid and a bioactive glass forms a bioactive crystalline phase when subjected to artificial saliva of Table 1A or 1B at 37° C. In some aspects, the formation of the bioactive crystalline phase takes place within 10 days, within 9 days, within 8 days, within 7 days, within 6 days, within 5 days, within 4 days, within 3 days, within 2 days, or within 1 day of subjecting at 37° C. to the artificial saliva of Table 1A or 1B or other aqueous compositions. In some aspects, the formation of the bioactive crystalline phase takes place within 7 days at 37° C. In some aspects, the formation of the bioactive crystalline phase takes place within 1 day at 37° C. In some aspects, a bioactive crystalline phase forms when subjected to real saliva (e.g., from an organism, such as a mammal, a human, and the like). In some aspects, the bioactive crystalline phase formation takes place in vivo, e.g., in real saliva in an organism, e.g., when brushing teeth of the organism or otherwise subjecting the teeth of the organism to a composition (e.g., comprising a bioactive glass and an amino acid) or to a bioactive glass.

In some aspects, the aqueous composition and/or artificial saliva comprises phosphate, calcium, fluoride, sodium, potassium, magnesium, a buffer, or any combination thereof. In some aspects, the aqueous composition and/or artificial saliva comprises potassium chloride, ammonium chloride, potassium dihydrogen phosphate, magnesium chloride hexahydrate, calcium chloride, HEPES buffer, sodium azide, sodium hydroxide, a combination thereof, or any combination thereof. In some aspects, the aqueous composition, such as artificial saliva, may include a salt solution comprising NaCl, NaHCO₃, KCl, K₂HPO₄, MgCl₂-6H₂O, CaCl₂), NaSO₄, (HOCH₂)₃CNH₂ in nano-pure water, with pH adjusted with acid, such as HCl. In some examples, the simulated body fluid comprises artificial saliva. In some aspects, an artificial saliva composition can comprise one or more of the components set forth below in Table 1A and 1B.

TABLE 1A

| Component | Wt. % Range |
|---|---|
| Potassium Chloride | 0.08-0.17 |
| Ammonium Chloride | 0.01-0.04 |
| Potassium Dihydrogen Phosphate | 0.03-0.08 |
| Magnesium Chloride Hexahydrate | 0.001-0.007 |
| Calcium Chloride | 0.007-0.023 |
| HEPES Buffer | 0.38-0.55 |
| Sodium Azide | 0.01-0.04 |
| Sodium Hydroxide | 0.001-0.025 |

TABLE 1B

| Component | Wt. % Specific Example |
|---|---|
| Potassium Chloride | 0.119 |
| Ammonium Chloride | 0.024 |
| Potassium Dihydrogen Phosphate | 0.054 |
| Magnesium Chloride Hexahydrate | 0.004 |
| Calcium Chloride | 0.011 |
| HEPES Buffer | 0.477 |
| Sodium Azide | 0.02 |
| Sodium Hydroxide | 0.012 |

Without wishing to be bound by theory, it is believed that one or more components of the artificial saliva, which has chemical composition similarities to real saliva, facilitates formation of a bioactive crystalline phase. For example, in some aspects, it is believed that one or more of phosphate, calcium, fluoride, sodium, potassium, magnesium, a buffer, or any combination thereof, but particularly the phosphate, calcium, fluoride, or a combination thereof, facilitates formation of a bioactive crystalline phase.

In some aspects, a bioactive crystalline phase is formed when subjected to the artificial saliva of Table 1A or Table 1B. In some aspects, the glass compositions herein form a bioactive crystalline phase when subjected to an aqueous composition and/or artificial saliva, wherein the aqueous composition and/or artificial saliva comprises one or more of potassium chloride, ammonium chloride, potassium dihydrogen phosphate, magnesium chloride hexahydrate, calcium chloride, HEPES buffer, sodium azide, sodium hydroxide, NaCl, NaHCO₃, NaSO₄, (HOCH₂)₃CNH₂ in nano-pure water, or any combination thereof. For example, the artificial saliva may comprise phosphate, calcium, fluoride, sodium, potassium, magnesium, a buffer, or any combination thereof.

In some aspects, the bioactive glass comprises a combination of SiO₂, CaO, and P₂O₅. In some aspects, the bioactive glass further comprises ZrO₂. In some aspects, the bioactive glass further comprises F⁻. In some aspects, the bioactive glass further also comprises MgO. In some aspects, the bioactive glass further comprises Li₂O, Na₂O, K₂O, or any combination thereof. In some aspects, the amount of Li₂O+Na₂O+K₂O is less than or equal to 5 wt. %.

In some aspects, the bioactive glass comprises SiO₂, CaO, and P₂O₅. In some aspects, the bioactive glass comprises SiO₂, CaO, P₂O₅, ZrO₂, and F⁻. In some aspects, when subjected to an aqueous composition, such as artificial saliva, such a bioactive glass forms a bioactive crystalline phase (e.g., within 7 days).

In some aspects, the bioactive glass comprises SiO₂ in an amount of 20-55 wt. %. In some aspects, the bioactive glass comprises SiO₂ in an amount of 15-55 wt. %. In some aspects, the bioactive glass comprises CaO in an amount of 20-55 wt. %. In some aspects, the bioactive glass comprises CaO in an amount of 20-60 wt. %. In some aspects, the bioactive glass comprises P₂O₅ in an amount of 5-30 wt. %. In some aspects, the bioactive glass comprises ZrO₂ in an amount of >0-15 wt. % (e.g., 0.1-15 wt. %). In some aspects, the bioactive glass comprises F⁻ in an amount of 0-5 wt. % (e.g., >0-5 wt. %).

In some aspects, the bioactive glass comprises:
15-55 wt. % SiO₂,
0.1-15 wt. % ZrO₂;
20-60 wt. % CaO, and
5-30 wt. % P₂O₅,
based on total weight of bioactive glass,
wherein, when the composition (e.g., containing bioactive glass and amino acid) is subjected to artificial saliva, a bioactive crystalline phase is formed (e.g., within 7 days or any of the other timeframes specified herein, such as within 1 day).

In some aspects, the bioactive glass comprises:
15-55 wt. % SiO₂,
0.1-15 wt. % ZrO₂,
20-60 wt. % CaO,
5-30 wt. % P₂O₅,
0-25 wt. % MgO,
0-10 wt. % SrO,
0-10 wt. % ZnO,
0-5 wt. % Al₂O₃,
0-5 wt. % B₂O₃,
0-5 wt. % F, and
0-5 wt. % Na₂O+K₂O+Li₂O,
based on total weight of bioactive glass,
wherein, when the composition (e.g., containing bioactive glass and amino acid) is subjected to artificial saliva, a bioactive crystalline phase is formed (e.g., within 7 days or any of the other timeframes specified herein, such as within 1 day).

In some aspects, the bioactive glass comprises:
25-55 wt. % SiO₂,
0.1-15 wt. % ZrO₂;
20-55 wt. % CaO,
5-30 wt. % P₂O₅,
0-10 wt. % MgO,
0-10 wt. % SrO,
0-10 wt. % ZnO,
0-5 wt. % Al₂O₃,
0-5 wt. % B₂O₃,
0-5 wt. % F, and
0-5 wt. % Na₂O+K₂O+Li₂O,
based on total weight of bioactive glass,
wherein, when the composition (e.g., containing bioactive glass and amino acid) is subjected to artificial saliva, a bioactive crystalline phase is formed (e.g., within 7 days or any of the other timeframes specified herein, such as within 1 day).

In some aspects, the bioactive glasses comprise:
25-35 wt. % SiO₂,
0.1-15 wt. % ZrO₂;
35-50 wt. % CaO,
15-25 wt. % P₂O₅,
0-25 wt. % MgO,
0-10 wt. % SrO,
0-10 wt. % ZnO,
0-5 wt. % Al₂O₃,
0-5 wt. % B₂O₃,
0-5 wt. % F, and
0-5 wt. % Na₂O+K₂O+Li₂O,
based on total weight of bioactive glass,
wherein, when the composition (e.g., containing bioactive glass and amino acid) is subjected to artificial saliva, a bioactive crystalline phase is formed (e.g., within 7 days or any of the other timeframes specified herein, such as within 1 day).

In some aspects, the bioactive glasses comprise:
15-45 wt. % SiO₂;
0.1-15 wt. % ZrO₂;
30-60 wt. % CaO; and
8-30 P₂O₅;
based on total weight of bioactive glass;
wherein, when the composition (e.g., containing bioactive glass and amino acid) is subjected to artificial saliva, a bioactive crystalline phase is formed (e.g., within 7 days or any of the other timeframes specified herein, such as within 1 day).

In some aspects, the bioactive glasses comprise:
15-45 wt. % SiO₂;
0.1-15 wt. % ZrO₂;
30-60 wt. % CaO;
8-30 P₂O₅;
0-25 wt. % MgO;
0-10 wt. % SrO;
0-10 wt. % ZnO;
0-5 wt. % Al₂O₃;
0-5 wt. % B₂O₃;
0-5 wt. % F⁻; and
0-5 wt. % Na₂O+K₂O+Li₂O;
based on total weight of bioactive glass;
wherein, when the composition (e.g., containing bioactive glass and amino acid) is subjected to artificial saliva, a bioactive crystalline phase is formed (e.g., within 7 days or any of the other timeframes specified herein, such as within 1 day).

The bioactivity is influenced by the composition of the glass.

Silicon dioxide (SiO₂) may serve as the primary glass-forming oxide component of the bioactive glasses disclosed herein. SiO₂ may be included to provide high temperature stability and chemical durability. However, if too much SiO₂ is included, such as a glass containing pure SiO₂, the melting temperature is too high to be readily workable (e.g., greater than 200 poise temperature). In addition, bioactive glasses including too much SiO₂ may suffer from decreased bioactivity.

In some aspects, the bioactive glasses comprise SiO₂ in an amount of at least 15 wt. % or at least 25 wt. %. In some aspects, the bioactive glasses comprise SiO₂ in an amount of 55 wt. % or less or 45 wt. % or less. In some aspects, the bioactive glasses comprise SiO₂ in an amount (wt. %) of 15-55, 15-50, 15-45, 15-40, 15-35, 15-30, 15-25, 15-20, 20-55, 20-50, 20-45, 20-40, 20-35, 20-30, 20-25, 25-55, 25-50, 25-45, 25-40, 25-35, 25-30, 30-55, 30-50, 30-45, 30-40, 30-35, 35-55, 35-50, 35-45, 35-40, 40-55,40-50, 40-45, 45-55, 45-50, or 50-55. In some aspects, the bioactive glass is substantially free of, or does not contain, SiO₂.

Phosphorus pentoxide (P₂O₅) may serve as a network former in bioactive glasses. Furthermore, the liberation of phosphate ions to the surface of bioactive glasses contributes to the formation of apatite. Apatite is an inorganic mineral in bone and teeth, and formation of apatite in a simulated body fluid, such as artificial saliva, is one criterion for a material to be bioactive, according to ASTM F1538-03 (2017), hereby incorporated by reference in its entirety for all purposes. The inclusion of phosphate ions in the bioactive glass increases apatite formation rate and the binding capacity of the hard tissues (e.g., bone, tooth, etc.). In addition, P₂O₅ can increase the viscosity of the glass, which in turn expands the range of operating temperatures, and is therefore an advantage to the manufacture and formation of the glass.

In some aspects, the bioactive glasses comprise P₂O₅ in an amount of at least 5 wt. %. In some aspects, the bioactive glasses comprise P₂O₅ in an amount of 30 wt. % or less. In some aspects, the bioactive glasses comprise P₂O₅ in an amount (wt. %) of 5-30, 5-25, 5-20, 5-15, 5-10, 5-8, 8-30, 8-25, 8-20, 8-15, 8-10, 10-30, 10-25, 10-20, 10-15, 15-30, 15-25, 15-20, 20-30, 20-25, or 25-30.

Zirconium dioxide (ZrO₂) may also be present and serves to function as a network former or intermediate in precursor glasses, as well as a key oxide for improving glass thermal stability by significantly reducing glass devitrification during forming and lowering liquidus temperature. In aspects, ZrO₂ may play a similar role as alumina (Al₂O₃) in the composition.

In some aspects, the bioactive glasses comprise ZrO₂ in an amount of 0 wt. % or greater than 0 wt. %. In some aspects, the bioactive glasses comprise ZrO₂ in an amount of 15 wt. % or less. In some aspects, the bioactive glasses comprise ZrO₂ in an amount (wt. %) of 0-15, 0-14, 0-13, 0-12, 0-11, 0-10, 0-9, 0-8, 0-7, 0-6, 0-5, 0-4, 0-3, 0-2, 0-0.8, 0-0.6, 0-0.4, 0-0.2, 0-0.1, >0-15, >0-14, >0-13, >0-12, >0-11, >0-10, >0-9, >0-8, >0-7, >0-6, >0-5, >0-4, >0-3, >0-2, >0-1, >0-0.8, >0-0.6, >0-0.4, >0-0.2, >0-0.1, 0.1-15, 0.1-14, 0.1-13, 0.1-12, 0.1-11, 0.1-10, 0.1-9, 0.1-8, 0.1-7, 0.1-6, 0.1-5, 0.1-4, 0.1-3, 0.1-2, 0.1-1, 0.1-0.8, 0.1-0.6, 0.1-0.4, 0.1-0.2, 0.2-15, 0.2-14, 0.2-13, 0.2-12, 0.2-11, 0.2-10, 0.2-9, 0.2-8, 0.2-7, 0.2-6, 0.2-5, 0.2-4, 0.2-3, 0.2-2, 0.2-1, 0.2-0.8, 0.2-0.6, 0.2-0.4, 0.4-15, 0.4-14, 0.4-13, 0.4-12, 0.4-11, 0.4-10, 0.4-9, 0.4-8, 0.4-7, 0.4-6, 0.4-5, 0.4-4, 0.4-3, 0.4-2, 0.4-1, 0.4-0.8, 0.4-0.6, 0.6-15, 0.6-14, 0.6-13, 0.6-12, 0.6-11, 0.6-10, 0.6-9, 0.6-8, 0.6-7, 0.6-6, 0.6-5, 0.6-4, 0.6-3, 0.6-2, 0.6-1, 0.6-0.8, 0.8-15, 0.8-14, 0.8-13, 0.8-12, 0.8-11, 0.8-10, 0.8-9, 0.8-8, 0.8-7, 0.8-6, 0.8-5, 0.8-4, 0.8-3, 0.8-2, 0.8-1, 1-15, 1-14, 1-13, 1-12, 1-11, 1-10, 1-9, 1-8, 1-7, 1-6, 1-5, 1-4, 1-3, 1-2, 2-15, 2-14, 2-13, 2-12, 2-11, 2-10, 2-9, 2-8, 2-7, 2-6, 2-5, 2-4, 2-3, 3-15, 3-14, 3-13, 3-12, 3-11, 3-10, 3-9, 3-8, 3-7, 3-6, 3-5, 3-4, 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, 5-6, 6-15, 6-4, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-4, 11-13, 11-12, 12-15, 12-4, 12-13, 13-15, 13-14, or 14-15. In some aspects, the bioactive glass is substantially free of, or does not contain, ZrO₂.

In some aspects, the bioactive glasses comprise fluoride (F⁻), which facilitates remineralization of enamel. Such remineralization can form bioactive crystalline phases, such as hydroxyapatite, fluorapatite, carbonated apatite, brushite, or any combination thereof. In some aspects, at least some of such bioactive crystalline phases are resistant to acid corrosion, such as fluorapatite. F⁻ can combine with CaO and P₂O₅ to form fluorapatite to improve the bioactivity of the bioactive glasses. Fluorapatite is an inorganic mineral in dental enamel. The ability to form fluorapatite can help regeneration of the enamel that has become demineralized, for example, due to tooth caries (e.g., cavities). In some aspects, formation of apatite ensures the high bioactivity of the compositions disclosed herein.

In some aspects, the F⁻ in the bioactive glasses is derived from calcium fluoride, magnesium fluoride, sodium fluoride, potassium fluoride, stannous fluoride, sodium monofluorophosphate, sodium difluorophosphate, or any combination thereof.

In some aspects, the bioactive glasses comprise F⁻ in an amount of 0 wt. % or greater than 0 wt. %. In some aspects, the bioactive glasses comprise F⁻ in an amount of 5 wt. % or less. In some aspects, the bioactive glasses comprise F⁻ in an amount (wt. %) of 0-5, 0-4.5, 0-4, 0-3.5, 0-3, 0-2.5, 0-2, 0-1.5, 0-1, 0-0.5, 0-0.1, >0-5, >0-4.5, >0-4, >0-3.5, >0-3, >0-2.5, >0-2, >0-1.5, >0-1, >0-0.5, >0-0.4, >0-0.3, >0-0.2, >0-0.1, >0-0.01, 0.01-5, 0.01-4.5, 0.01-4, 0.01-3.5, 0.01-3, 0.01-2.5, 0.01-2, 0.01-1.5, 0.01-1, 0.01-0.5, 0.01-0.4, 0.01-0.3, 0.01-0.2, 0.01-0.1, 0.1-5, 0.1-4.5, 0.1-4, 0.1-3.5, 0.1-3, 0.1-2.5, 0.1-2, 0.1-1.5, 0.1-1, 0.1-0.5, 0.1-0.4, 0.1-0.3, 0.1-0.2, 0.1-0.15, 0.2-5, 0.2-4.5, 0.2-4, 0.2-3.5, 0.2-3, 0.2-2.5, 0.2-2, 0.2-1.5, 0.2-1, 0.2-0.5, 0.2-0.4, 0.2-0.3, 0.3-5, 0.3-4.5, 0.3-4, 0.3-3.5, 0.3-3, 0.3-2.5, 0.3-2, 0.3-1.5, 0.3-1, 0.3-1.5, 0.3-0.4, 0.4-5, 0.4-4.5, 0.4-4, 0.4-3.5, 0.4-3, 0.4-2.5, 0.4-2, 0.4-1.5, 0.4-1, 0.4-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5. In some aspects, the bioactive glass is substantially free of, or does not contain, F⁻.

Divalent cation oxides (such as alkaline earth oxides and ZnO) improve the melting behavior, chemical durability, and bioactivity of the glass. In addition, alkaline earth oxides may improve other desirable properties in the materials, including influencing the Young's modulus and the coefficient of thermal expansion. In examples, the bioactive glasses comprise from 30-70 wt. % MO, wherein MO is the sum of MgO, CaO, SrO, BeO, and BaO. In some aspects, the bioactive glasses comprise MO in an amount (wt. %) of 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the bioactive glasses comprise CaO in an amount of at least 20 wt. %. In some aspects, the bioactive glasses comprise CaO in an amount of 60 wt. % or less or 55 wt. % or less. In some aspects, the bioactive glasses comprise CaO in an amount (wt. %) of 20-60, 20-55, 20-50, 20-45, 20-40, 20-35, 20-30, 20-25, 25-60, 25-55, 25-50, 25-45, 25-40, 25-35, 25-30, 30-60, 30-55, 30-50, 30-45, 30-40, 30-35, 35-60, 35-55, 35-50, 35-45, 35-40, 40-60, 40-55, 40-50, 40-45, 45-60, 45-55, 45-50, 50-60, 50-55, or 55-60. In some aspects, the bioactive glass is substantially free of, or does not contain, CaO.

In some aspects, the bioactive glasses comprise MgO. In some aspects, the inclusion of MgO can improve liquidus of the precursor glass to avoid devitrification during forming. In some aspects, the bioactive glasses comprise MgO in an amount of 0 wt. % or greater than 0 wt. %. In some aspects, the bioactive glasses comprise MgO in an amount of 25 wt. % or less or 10 wt. % or less. In some aspects, the bioactive glasses comprise MgO in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. In some aspects, the bioactive glasses comprise MgO in an amount (wt. %) of 0-25, 0-20, 0-15, 0-10, 0-5, 0-1, >0-25, >0-20, >0-15, >0-10, >0-5, >0-1, 1-25, 1-20, 1-15, 1-10, 1-5, 5-25, 5-20,5-15, 5-10, 10-25, 10-20, 10-15, 15-25, 15-20, or 20-25. In some aspects, the bioactive glass is substantially free of, or does not contain, MgO.

In some aspects, the bioactive glasses comprise SrO. In some aspects, the inclusion of SrO can improve liquidus of the precursor glass to avoid devitrification during forming. Moreover, in some aspects, SrO may also enter the structure of apatite to improve bioactivity. In some aspects, the bioactive glasses comprise SrO in an amount of 0 wt. % or greater than 0 wt. %. In some aspects, the bioactive glasses comprise SrO in an amount of 10 wt. % or less. In some aspects, the bioactive glasses comprise SrO in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. In some aspects, the bioactive glass is substantially free of, or does not contain, SrO.

In some aspects, the bioactive glasses comprise ZnO. In some aspects, the inclusion of ZnO can improve liquidus of the precursor glass to avoid devitrification during forming. Moreover, in some aspects, ZnO may also enter the structure of apatite to improve bioactivity. In some aspects, the bioactive glasses comprise ZnO in an amount of 0 wt. % or greater than 0 wt. %. In some aspects, the bioactive glasses comprise ZnO in an amount of 10 wt. % or less. In some aspects, the bioactive glasses comprise ZnO in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. In some aspects, the bioactive glass is substantially free of, or does not contain, ZnO.

In some aspects, CaO is found to be able to react with P₂O₅ to form apatite when immersed in a simulated body fluid (SBF), such as artificial saliva, or in vivo or in real saliva. The release of Ca²⁺ ions from the surface of the glass contributes to the formation of a layer rich in calcium phosphate. Thus, the combination of P₂O₅ and CaO may provide advantageous compositions for bioactive glasses.

In some aspects, the bioactive glasses comprise the sum P₂O₅+CaO in an amount of 40 wt. % or more. In some aspects, the bioactive glasses comprise the sum P₂O₅+CaO in an amount of 70 wt. % or less. In some aspects, the bioactive glasses comprise the sum P₂O₅+CaO in an amount (wt. %) of 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the bioactive glasses comprise the sum MgO+CaO in an amount of at least 20 wt. %. In some aspects, the bioactive glasses comprise the sum MgO+CaO in an amount of 70 wt. % or less. In some aspects, the bioactive glasses comprise the sum MgO+CaO in an amount (wt. %) of20-70, 20-65, 20-60, 20-55, 20-50, 20-45, 20-40, 20-35, 20-30, 20-25, 25-70, 25-65, 25-60, 25-55, 25-50, 25-45, 25-40, 25-35, 25-30, 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the bioactive glasses comprise the sum F⁻+P₂O₅ in an amount of at least 5 wt. %. In some aspects, the bioactive glasses comprise the sum F⁻+P₂O₅ in an amount of 35 wt. % or less. In some aspects, the bioactive glasses comprise the sum F⁻+P₂O₅ in an amount (wt. %) of 5-35, 5-30, 5-25, 5-20, 5-15, 5-10, 10-35, 10-30, 10-25, 10-20, 10-15, 15-35, 15-30, 15-25, 15-20, 20-35, 20-30, 20-25, 25-35, or 25-30.

In some aspects, the bioactive glasses comprise B₂O₃ in an amount of 0 wt. % or greater than 0 wt. %. In some aspects, the bioactive glasses comprise B₂O₃ in an amount of 5 wt. % or less. In some aspects, the bioactive glasses comprise B₂O₃ in an amount (wt. %) of 0-5, 0-4.5, 0-4, 0-3.5, 0-3, 0-2.5, 0-2, 0-1.5, 0-1, 0-0.5, 0-0.4, 0-0.3, 0-0.2, 0-0.1, 0-0.05, >0-5, >0-4.5, >0-4, >0-3.5, >0-3, >0-2.5, >0-2, >0-1.5, >0-1, >0-0.5, >0-0.4, >0-0.3, >0-0.2, >0-0.1, >0-0.05, 0.05-5, 0.05-4.5, 0.05-4, 0.05-3.5, 0.05-3, 0.05-2.5, 0.05-2, 0.05-1.5, 0.05-1, 0.05-0.5, 0.05-0.4, 0.05-0.3, 0.05-0.2, 0.05-0.1, 0.1-5, 0.1-4.5, 0.1-4, 0.1-3.5, 0.1-3, 0.1-2.5, 0.1-2, 0.1-1.5, 0.1-1, 0.1-0.5, 0.1-0.4, 0.1-0.3, 0.1-0.2, 0.2-5, 0.2-4.5, 0.2-4, 0.2-3.5, 0.2-3, 0.2-2.5, 0.2-2, 0.2-1.5, 0.2-1, 0.2-0.5, 0.2-0.4, 0.2-0.3, 0.3-5, 0.3-4.5, 0.3-4, 0.3-3.5, 0.3-3, 0.3-2.5, 0.3-2, 0.3-1.5, 0.3-1, 0.3-0.5, 0.3-0.4, 0.4-5, 0.4-4.5, 0.4-4, 0.4-3.5, 0.4-3, 0.4-2.5, 0.4-2, 0.4-1.5, 0.4-1, 0.4-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5. In some aspects, the bioactive glass is substantially free of, or does not contain, B₂O₃.

In some aspects, the bioactive glasses can comprise Al₂O₃. In some aspects, Al₂O₃ may influence (e.g., stabilize) the network structure of the glass, improve mechanical properties, improve chemical durability, or any combination thereof. In some aspects, Al₂O₃ may lower liquidus temperature, lower coefficient of thermal expansion, enhance the strain point, or any combination thereof. In some aspects, in addition to its role as a network former, Al₂O₃(and ZrO₂) may help improve the chemical durability and mechanical properties in silicate glass while having no toxicity concerns. In some aspects, too high a content of Al₂O₃ or ZrO₂ (e.g., >20 wt. %) generally increases the viscosity of the melt and decreases bioactivity.

In some aspects, the bioactive glasses comprise Al₂O₃ in an amount of 0 wt. % or greater than 0 wt. %. In some aspects, the bioactive glasses comprise Al₂O₃ in an amount of 5 wt. % or less. In some aspects, the bioactive glasses comprise Al₂O₃ in an amount (wt. %) of 0-5, 0-4.5, 0-4, 0-3.5, 0-3, 0-2.5, 0-2, 0-1.5, 0-1, 0-0.5, 0-0.4, 0-0.3, 0-0.2, 0-0.1, 0-0.05, >0-5, >0-4.5, >0-4, >0-3.5, >0-3, >0-2.5, >0-2, >0-1.5, >0-1, >0-0.5, >0-0.4, >0-0.3, >0-0.2, >0-0.1, >0-0.05, 0.05-5, 0.05-4.5, 0.05-4, 0.05-3.5, 0.05-3, 0.05-2.5, 0.05-2, 0.05-1.5, 0.05-1, 0.05-0.5, 0.05-0.4, 0.05-0.3, 0.05-0.2, 0.05-0.1, 0.1-5, 0.1-4.5, 0.1-4, 0.1-3.5, 0.1-3, 0.1-2.5, 0.1-2, 0.1-1.5, 0.1-1, 0.1-0.5, 0.1-0.4, 0.1-0.3, 0.1-0.2, 0.2-5, 0.2-4.5, 0.2-4, 0.2-3.5, 0.2-3, 0.2-2.5, 0.2-2, 0.2-1.5, 0.2-1, 0.2-0.5, 0.2-0.4, 0.2-0.3, 0.3-5, 0.3-4.5, 0.3-4, 0.3-3.5, 0.3-3, 0.3-2.5, 0.3-2, 0.3-1.5, 0.3-1, 0.3-0.5, 0.3-0.4, 0.4-5, 0.4-4.5, 0.4-4, 0.4-3.5, 0.4-3, 0.4-2.5, 0.4-2, 0.4-1.5, 0.4-1, 0.4-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5. In some aspects, the bioactive glass is substantially free of, or does not contain, Al₂O₃.

In some aspects, alkali oxides (Na₂O, K₂O, Li₂O, Rb₂O, or Cs₂O serve as aids in achieving low melting temperature and low liquidus temperatures, which can aid glass melting processes. In addition, in some aspects, the addition of alkali oxides can improve bioactivity. However, in some aspects, if the amount of alkali oxides is too high, the bioactive glasses have reduced chemical durability; in other words, in some aspects, keeping the amount of alkali oxides to a certain lower range achieves high chemical durability of the bioactive glasses. In some aspects, the amount of alkali oxides, such as Na₂O+K₂O+Li₂O, is minimized or at least kept to an amount below 10 wt. % or below 5 wt. %, so as to improve durability of the bioactive glass in aqueous compositions, such as in water. In some aspects, the bioactive glasses do not contain any alkali oxides (e.g., do not contain any added alkali oxides). In some aspects, the bioactive glasses are substantially free of alkali oxides (e.g., contain less than 0.1 wt. % alkali oxides).

In some aspects, the bioactive glasses comprise Li₂O in an amount of 0 wt. % or greater than 0 wt. %. In some aspects, the bioactive glasses comprise Li₂O in an amount of 10 wt. % or less. In some aspects, the bioactive glasses comprise Li₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the bioactive glasses comprise Na₂O in an amount of 0 wt. % or greater than 0 wt. %. In some aspects, the bioactive glasses comprise Na₂O in an amount of 10 wt. % or less. In some aspects, the bioactive glasses comprise Na₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the bioactive glasses comprise K₂O in an amount of 0 wt. % or greater than 0 wt. %. In some aspects, the bioactive glasses comprise K₂O in an amount of 10 wt. % or less. In some aspects, the bioactive glasses comprise K₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the bioactive glasses comprise the sum Li₂O+Na₂O+K₂O in an amount of 0 wt. % or greater than 0 wt. %. In some aspects, the bioactive glasses comprise the sum Li₂O+Na₂O+K₂O in an amount of 10 wt. % or less or 5 wt. % or less. In some aspects, the bioactive glasses comprise the sum Li₂O+Na₂O+K₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. In some aspects, the bioactive glasses comprise the sum Na₂O+K₂O+Li₂O+Rb₂O+Cs₂O in any of the amounts set forth in this paragraph.

In some aspects, additional components can be incorporated into the bioactive glasses to provide additional benefits or may be incorporated as contaminants typically found in commercially-prepared glass. For example, additional components can be added as coloring or fining agents (e.g., to facilitate removal of gaseous inclusions from melted batch materials used to produce the bioactive glasses) and/or for other purposes. In some aspects, the bioactive glasses may comprise one or more compounds useful as ultraviolet radiation absorbers. In some aspects, the bioactive glasses can comprise 3 wt. % or less ZnO, TiO₂, CeO, MnO, Nb₂O₅, MoO₃, Ta₂O₅, WO₃, SnO₂, Fe₂O₃, As₂O₃, Sb₂O₃, Cl, Br, or any combination thereof. In some aspects, the bioactive glasses can comprise from 0 to about 3 wt. %, 0 to about 2 wt. %, 0 to about 1 wt. %, 0 to 0.5 wt. %, 0 to 0.1 wt. %, 0 to 0.05 wt. %, or 0 to 0.01 wt. % ZnO, TiO₂, CeO, MnO, Nb₂O₅, MoO₃, Ta₂O₅, WO₃, SnO₂, Fe₂O₃, As₂O₃, Sb₂O₃, Cl, Br, or any combination thereof. In some aspects, the bioactive glasses can also include various contaminants associated with batch materials and/or introduced into the glass by the melting, fining, and/or forming equipment used to produce the glass. For example, in some aspects, the bioactive glasses can comprise from 0 to about 3 wt. %, 0 to about 2 wt. %, 0 to about 1 wt. %, 0 to about 0.5 wt. %, 0 to about 0.1 wt. %, 0 to about 0.05 wt. %, or 0 to about 0.01 wt. % SnO₂ or Fe₂O₃, or a combination thereof.

In some aspects, the bioactive glass is substantially free of, or does not contain, a crystalline phase. For example, in some aspects, the bioactive glass has a glassy phase and no crystalline phase. In some aspects, the bioactive glass is a glass (e.g., a glass article). In some aspects, the bioactive glass contains a crystalline phase. In some aspects, the bioactive glass is a glass-ceramic (e.g., a glass-ceramic article).

In some aspects, the bioactive glass is in a form of particulates, microbeads, fibers, or a combination thereof. In some aspects, the particulates are a fine powder. Methods for preparing particulates, microbeads, and/or fibers from bioactive glasses is known in the art. In some aspects, the particulates, microbeads, and/or fibers have an average particle size of at least 1 micron. In some aspects, the particulates and/or microbeads have an average particle size of 50 microns or less. In some aspects, the particulates and/or microbeads have an average particle size (microns) of 1-50, 1-45, 1-40, 1-35, 1-30, 1-25, 1-20, 1-15, 1-10, 1-5, 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50. Average particle size, as used herein, means the average size as measured by dynamic light scattering using commercially available equipment.

In some aspects, when subjected to an aqueous composition (e.g., artificial saliva or real saliva), as described elsewhere herein, a bioactive crystalline phase is formed, e.g., on a tooth and/or on a surface of the bioactive glass. In some aspects, the formation of the bioactive crystalline phase takes place within a certain timeframe, such as within 1, 2, 3, 4, 5, 6, 7, or 8 days, as described elsewhere herein. In some aspects, the bioactive crystalline phase comprises apatite, brushite, or a combination thereof. In some aspects, the apatite comprises hydroxyapatite, fluorapatite, carbonated apatite, or any combination thereof. In some aspects, the brushite is a precursor to apatite.

In some aspects, disclosed is a composition, comprising:
an amino acid; and
a bioactive glass, comprising:
   15-55 wt. % SiO₂;
   0.1-15 wt. % ZrO₂;
   20-60 wt. % CaO; and
   5-30 wt. % P₂O₅;
   based on total weight of bioactive glass;
   wherein, when the composition is subjected to an aqueous composition, a bioactive crystalline phase is formed within 7 days (or any of the other timeframes specified herein, such as within 1 day).

In some aspects, the bioactive glass is present in the composition in an amount of 0 wt. % or greater than 0 wt. %. In some aspects, the bioactive glass is present in the composition in an amount of 20 wt. % or less. aspects, the bioactive glass is present in the composition in an amount (wt. %) of 0-20, 0-18, 0-16, 0-14, 0-12, 0-10, 0-8, 0-6, 0-5, 0-4, 0-3, 0-2, 0-1, 0-0.8, 0-0.6, 0-0.4, 0-0.2, 0-0.1, >0-20, >0-18, >0-16, >0-14, >0-12, >0-10, >0-8, >0-6, >0-5, >0-4, >0-3, >0-2, >0-1, >0-0.8, >0-0.6, >0-0.4, >0-0.2, >0-0.1, 0.1-20, 0.1-18, 0.1-16, 0.1-14, 0.1-12, 0.1-10, 0.1-8, 0.1-6, 0.1-5, 0.1-4, 0.1-3, 0.1-2, 0.1-1, 0.1-0.8, 0.1-0.6, 0.1-0.4, 0.1-0.2, 0.2-20, 0.2-18, 0.2-16, 0.2-14, 0.2-12, 0.2-10, 0.2-8, 0.2-6, 0.2-5, 0.2-4, 0.2-3, 0.2-2, 0.2-1, 0.2-0.8, 0.2-0.6, 0.2-0.4, 0.4-20, 0.4-18, 0.4-16, 0.4-14, 0.4-12, 0.4-10, 0.4-8, 0.4-6, 0.4-5, 0.4-4, 0.4-3, 0.4-2, 0.4-1, 0.4-0.8, 0.4-0.6, 0.6-20, 0.6-18, 0.6-16, 0.6-14, 0.6-12, 0.6-10, 0.6-8, 0.6-6, 0.6-5, 0.6-4, 0.6-3, 0.6-2, 0.6-1, 0.6-0.8, 0.8-20, 0.8-18, 0.8-16, 0.8-14, 0.8-12, 0.8-10, 0.8-8, 0.8-6, 0.8-5, 0.8-4, 0.8-3, 0.8-2, 0.8-1, 1-20, 1-18, 1-16, 1-14, 1-12, 1-10, 1-8, 1-6, 1-5, 1-4, 1-3, 1-2, 2-20, 2-18, 2-16, 2-14, 2-12, 2-10, 2-8, 2-6, 2-5, 2-4, 2-3, 3-20, 3-18, 3-16, 3-14, 3-12, 3-10, 3-8, 3-6, 3-5, 3-4, 4-20, 4-18, 4-16, 4-14, 4-12, 4-10, 4-8, 4-6, 4-5, 5-20, 5-18, 5-16, 5-14, 5-12, 5-10, 5-8, 5-6, 6-20, 6-18, 6-16, 6-14, 6-12, 6-10, 6-8, 8-20, 8-18, 8-16, 8-14, 8-12, 8-10, 10-20, 10-18, 10-16,10-14, 10-12, 12-20, 12-18, 12-16, 12-14, 14-20, 14-18, 14-16, 16-20, 16-18, or 18-20.

In some aspects, the amino acid is present in the composition in an amount of 0 wt. % or greater than 0 wt. %. In some aspects, the amino acid is present in the composition in an amount of 25 wt. % or less. In some aspects, the amino acid is present in the composition in an amount (wt. %) of 0-25, 0-24, 0-22, 0-20, 0-18, 0-16, 0-14, 0-12, 0-10, 0-9, 0-8, 0-7, 0-6, 0-5, 0-4, 0-3, 0-2, 0-1, 0-0.5, 0-0.1, >0-25, >0-24, >0-22, >0-20, >0-18, >0-16, >0-14, >0-12, >0-10, >0-9, >0-8, >0-7, >0-6, >0-5, >0-4, >0-3, >0-2, >0-1, >0-0.5, >0-0.1, 0.1-25, 0.1-24, 0.1-22, 0.1-20, 0.1-18, 0.1-16, 0.1-14, 0.1-12, 0.1-10, 0.1-9, 0.1-8, 0.1-7, 0.1-6, 0.1-5, 0.1-4, 0.1-3, 0.1-2, 0.1-1, 0.1-0.5, 0.5-25, 0.5-24, 0.5-22, 0.5-20, 0.5-18, 0.5-16, 0.5-14, 0.5-12, 0.5-10, 0.5-9, 0.5-8, 0.5-7, 0.5-6, 0.5-5, 0.5-4, 0.5-3, 0.5-2, 0.5-1, 1-25, 1-24, 1-22, 1-20, 1-18, 1-16, 1-14, 1-12, 1-10, 1-9, 1-8, 1-7, 1-6, 1-5, 1-4, 1-3, 1-2, 2-25, 2-24, 2-22, 2-20, 2-18, 2-16, 2-14, 2-12, 2-10, 2-9, 2-8, 2-7, 2-6, 2-5, 2-4, 2-3, 3-25, 3-24, 3-22, 3-20, 3-18, 3-16, 3-14, 3-12, 3-10, 3-9, 3-8, 3-7, 3-6, 3-5, 3-4, 4-25, 4-24, 4-22, 4-20, 4-18, 4-16, 4-14, 4-12, 4-10, 4-9, 4-8, 4-7, 4-6, 4-5, 5-25, 5-24, 5-22, 5-20, 5-18, 5-16, 5-14, 5-12, 5-10, 5-9, 5-8, 5-7, 5-6, 6-25, 6-24, 6-22, 6-20, 6-18, 6-16, 6-14, 6-12, 6-10, 6-9, 6-8, 6-7, 7-25, 7-24, 7-22, 7-20, 7-18, 7-16, 7-14, 7-12, 7-10, 7-9, 7-8, 8-25, 8-24, 8-22, 8-20, 8-18, 8-16, 8-14, 8-12, 8-10, 8-9, 9-25, 9-24, 9-22, 9-20, 9-18, 9-16, 9-14, 9-12, 9-10, 10-25, 10-24, 10-22, 10-20, 10-18, 10-16, 10-14, 10-12, 12-25, 12-24, 12-22, 12-20, 12-18, 12-16, 12-14, 14-25, 14-24, 14-22, 14-20, 14-18, 14-16, 16-25, 16-24, 16-22, 16-20, 16-18, 18-25, 18-24, 18-22, 18-20, 20-25, 20-24, 20-22, 22-25, 22-24, or 24-25. In some aspects, the composition is substantially free of, or does not contain, amino acid (e.g., any amino acid or any specific amino acid or combination of specific amino acids disclosed elsewhere herein). In some aspects, the amino acid is present in the composition in an amount of 5-15 wt. % or 4-16 wt. %.

In some aspects, the amino acid (e.g., that is present in the composition) is glycine, glutamic acid, arginine, histidine, lysine, pyrrolysine, aspartic acid, serine, threonine, asparagine, glutamine, cysteine, selenocysteine, proline, alanine, valine, isoleucine, leucine, methionine, phenylalanine, tyrosine, tryptophan, or any combination thereof. In some aspects, the amino acid is glycine, glutamic acid, or a combination thereof. In some aspects, the amino acid is histidine, lysine, pyrrolysine, aspartic acid, serine, threonine, asparagine, glutamine, cysteine, selenocysteine, proline, alanine, valine, isoleucine, leucine, methionine, phenylalanine, tyrosine, tryptophan, or any combination thereof. In some aspects, the amino acid is arginine. In some aspects, the amino acid is not arginine. In some aspects, the amino acid is not one or more of glycine, glutamic acid, arginine, histidine, lysine, pyrrolysine, aspartic acid, serine, threonine, asparagine, glutamine, cysteine, selenocysteine, proline, alanine, valine, isoleucine, leucine, methionine, phenylalanine, tyrosine, tryptophan, or any combination thereof.

In some aspects, the bioactive glasses can be made via traditional methods. For example, in some aspects, precursor glasses can be formed by thoroughly mixing the requisite batch materials (for example, using a Turbula® mixer) in order to secure a homogeneous melt, and subsequently placing into silica and/or platinum crucibles. The crucibles can be placed into a furnace and the glass batch melted and maintained at temperatures ranging from 1100° C. to 1400° C. for times ranging from about 6 hours to 24 hours. The melts can thereafter be poured into steel molds to yield glass slabs. Subsequently, those slabs optionally can be transferred immediately to an annealer operating at about 400° C. to 700° C., where the glass is held at temperature for about 0.5 hour to 3 hours and subsequently cooled overnight. In another non-limiting example, precursor glasses are prepared by dry blending the appropriate oxides and mineral sources for a time sufficient to thoroughly mix the ingredients. The glasses are melted in platinum crucibles at temperatures ranging from about 1100° C. to 1400° C. and held at temperature for about 6 hours to 16 hours. The resulting glass melts are then poured onto a steel table to cool. The precursor glasses optionally are then annealed at appropriate temperatures.

In some aspects, the resulting bioactive glasses can be ground into fine particles in the range of 1-10 microns (m) by air jet milling. The particle size can be varied in the range of 1-100 m using attrition milling or ball milling of glass frits. Furthermore, these glasses can be processed into short fibers, beads, sheets or three-dimensional scaffolds using different methods. Short fibers are made by melt spinning or electric spinning; beads can be produced by flowing glass particles through a hot vertical furnace or a flame torch; sheets can be manufactured using thin rolling, float or fusion-draw processes; and scaffolds can be produced using rapid prototyping, polymer foam replication and particle sintering. Glasses of desired forms can be used to support cell growth, soft and hard tissue regeneration, stimulation of gene expression or angiogenesis.

Continuous fibers can be easily drawn from the disclosed composition using processes known in the art. For example, fibers can be formed using a directly heated (electricity passing directly through) platinum bushing. Glass cullet is loaded into the bushing, heated up until the glass can melt. Temperatures are set to achieve a desired glass viscosity (usually <1000 poise) allowing a drip to form on the orifice in the bushing (Bushing size is selected to create a restriction that influences possible fiber diameter ranges). The drip is pulled by hand to begin forming a fiber. Once a fiber is established it is connected to a rotating pulling/collection drum to continue the pulling process at a consistent speed. Using the drum speed (or revolutions per minute RPM) and glass viscosity the fiber diameter can be manipulated—in general the faster the pull speed, the smaller the fiber diameter. Glass fibers with diameters in the range of 1-100 μm can be drawn continuously from a glass melt. Fibers can also be created using an updraw process. In this process, fibers are pulled from a glass melt surface sitting in a box furnace. By controlling the viscosity of the glass, a quartz rod is used to pull glass from the melt surface to form a fiber. The fiber can be continuously pulled upward to increase the fiber length. The velocity that the rod is pulled up determines the fiber thickness along with the viscosity of the glass.

Therefore, in some aspects, a method for making the bioactive glasses disclosed herein is provided, the method comprising:
combining each component of the bioactive glass to form a mixture,
heating the mixture to a temperature of 1500° C. or less to form a melted mixture, and
cooling the melted mixture to form a cooled mixture.

In some aspects, the method further comprising forming the cooled mixture into particulates, microbeads, fibers, or a combination thereof.

Aspects are related to compositions containing bioactive glasses (e.g., and one or more amino acids), and the methods of using the compositions to treat medical conditions. The compositions can be in a form of a toothpaste, mouthwash, gel, varnish, rinse, spray, ointment, salve, cream, bandage, polymer film, oral formulation, pill, capsule, transdermal formulation, dental strips (e.g., containing gel, oral formulation, etc.), dental tray (e.g., containing gel, oral formulation, etc.), or any combination thereof. In some aspects, the composition is a dentifrice composition. The bioactive glasses and/or amino acid(s) can be physically or chemically attached to the compositions or simply mixed in. As noted above, the bioactive glass can be in any form that works in the application, including particles, beads, particulates, short fibers, long fibers, or woolen meshes. The methods of using the composition (e.g., containing bioactive glass and amino acid) to treat a medical condition can be simply like the use of composition as normally applied.

In some aspects, disclosed is a composition comprising the bioactive glasses. In some aspects, the composition comprises a bioactive glass, wherein the bioactive glass is attached to the composition or mixed therein. In some aspects, the composition further comprises an amino acid (e.g., one or more amino acids). In some aspects, the composition is in a form of a toothpaste, mouthwash, gel, varnish, rinse, spray, ointment, salve, cream, bandage, polymer film, oral formulation, pill, capsule, transdermal formulation, dental strips (e.g., containing gel, oral formulation, etc.), dental tray (e.g., containing gel, oral formulation, etc.), or any combination thereof.

In some aspects, the composition is substantially free of, or does not include, a fluoride ion source that is derived from the bioactive glass (e.g., a fluoride ion source that is added to the composition as a component separate from the bioactive glass and which fluoride ion source is not derived from the bioactive glass). In this regard, in some aspects, the composition does not contain calcium fluoride, magnesium fluoride, sodium fluoride, potassium fluoride, stannous fluoride, sodium monofluorophosphate, sodium difluorophosphate, or any combination thereof. In some aspects, the composition does not contain a source of fluoride ions that is added to the composition separately from the bioactive glass. In some aspects, the composition includes a fluoride ion source that is added separately to the composition. In some aspects, the composition comprises calcium fluoride, magnesium fluoride, sodium fluoride, potassium fluoride, stannous fluoride, sodium monofluorophosphate, sodium difluorophosphate, or any combination thereof. In some aspects, the calcium fluoride, magnesium fluoride, sodium fluoride, potassium fluoride, stannous fluoride, sodium monofluorophosphate, sodium difluorophosphate, or any combination thereof are added to the composition separately from the bioactive glass.

In some aspects, the composition is or comprises a dentifrice composition. In some aspects, the dentifrice composition is a toothpaste, a mouthwash, or a gel, or any other formulation that is intended for oral care (e.g., for use in dental strips or dental trays). In some aspects, without wishing to be bound by theory, it is believed that high chemical durability and high bioactivity of the bioactive glasses makes them advantageous for use in oral care formulations (e.g., dentifrice compositions) or cosmetic formulations.

In some aspects, the composition is or comprises a skin care or cosmetic composition. In some aspects, the skin care or cosmetic composition is a gel, a spray, an ointment, a salve, a cream, a lotion, a bandage, a transdermal formulation, or any other formulation that is intended for skin care. In some aspects, without wishing to be bound by theory, it is believed that high chemical durability and high bioactivity of the bioactive glasses makes them advantageous for use in oral care formulations (e.g., dentifrice compositions), skin care, or cosmetic formulations.

In some aspects, the dentifrice, skin care, or cosmetic compositions can comprise any suitable components and amounts of such components. For example, in some aspects, the dentifrice, skin care, or cosmetic compositions comprise the bioactive glass in combination with one or more of an amino acid, a solvent, a humectant, a fluoride ion source, a flavoring, a surfactant, a sweetener, a whitening agent, an abrasive, a thickening/gelling agent, a pH/buffer, a preservative, or any combination thereof.

In some aspects, the composition is a dentifrice composition intended to be delivered to the surface(s) of one or more teeth via dental strips or dental trays. In some aspects, the dentifrice composition can be applied to a strip (e.g., flexible or rigid strip) or tray (e.g., flexible or rigid) at a manufacturing facility thereby forming a dental strip or dental tray containing the composition, and then selling or otherwise providing the dental strip or dental tray to an end user (e.g., a consumer or a dental professional), or the end user may apply the dentifrice composition to a strip or tray prior to applying the dental strip or dental tray (now containing the dentifrice composition) to one or more teeth (e.g., the end user's teeth or another's teeth). In some aspects, the strips are plastic, paper, or another material sufficiently stable to the conditions of application, storage, and use and which conforms sufficiently to one or more teeth so as to bring the dentifrice composition contained on the strip into contact with at least a portion of one or more teeth.

Such dentifrice, skin care, or cosmetic compositions may be aqueous based or they may be non-aqueous based. When aqueous based, the solvent comprises water. When non-aqueous based, the solvent typically comprises glycerin (for dentifrice, skin care, or cosmetic compositions), or an oil or a polymer (for skin care or cosmetic compositions). In some aspects, a suitable dentifrice, skin care, or cosmetic composition with ranges of components is shown in Table 2A. Notably, as indicated in Table 2A, there is overlap between components suitable, for example, for a dentifrice composition and those suitable for a skin care or cosmetic composition.

TABLE 2A

| Component | Amount (wt. %) |
|---|---|
| Bioactive glass | >0-20 |
| Amino Acid(s) | 0-25 |
| Solvent | 10-80 |
| Humectant | 0-80 |
| Fluoride Ion Source | 0-1 |
| Flavoring | 0-0.5 |
| Surfactant | 0-5 |
| Sweetener | 0-5 |
| Whitening Agent | 0-2 |
| Abrasive | 0-20 |
| Thickening/Gelling Agent | 0-15 |
| pH Modifier/Buffer | 0-25 |
| Preservative | 0-0.1 |
| Other | 0-10 |

In some aspects, the dentifrice composition is present on/in (or is intended for or configured for) a dental strip or in a dental tray. In some aspects, the dentifrice composition is that shown in Table 2A, Table 2B, or any other composition disclosed elsewhere herein (or that can be formed from the disclosures herein), for example, in instances where the dentifrice composition is present on/in a dental strip of dental tray, though other uses for the composition of Table 2B are contemplated as well, such as toothpaste, mouthwash, etc.

TABLE 2B

| Component | Amount (wt. %) |
|---|---|
| Bioactive glass | 0.5-15 |
| Amino acid | 0.1-25 |
| Solvent | 10-80 |
| Humectant | 0.0001-80 |
| Thickening/Gelling Agent | 0.01-15 |
| pH Modifier/Buffer | 0.0001-25 |
| Preservative | 0.015-0.1 |
| Flavoring/Sweetener | 0.001-5 |

In some aspects, the dentifrice, skin care, or cosmetic compositions comprise the bioactive glass in amount of greater than 0 wt. %. In some aspects, the dentifrice, skin care, or cosmetic compositions comprise the bioactive glass in amount of 20 wt. % or less. In some aspects, the dentifrice, skin care, or cosmetic compositions comprise the bioactive glass in amount (wt. %) of >0-20, >0-18, >0-16, >0-14, >0-12, >0-10, >0-8, >0-6, >0-4, >0-2, >0-1, >0-0.5, >0-0.1, 0.1-20, 0.1-18, 0.1-16, 0.1-14, 0.1-12, 0.1-10, 0.1-8, 0.1-6, 0.1-4, 0.1-2, 0.1-1, 0.1-0.5, 0.5-20, 0.5-18, 0.5-16, 0.5-14, 0.5-12, 0.5-10, 0.5-8, 0.5-6, 0.5-4, 0.5-2, 0.5-1, 1-20, 1-18, 1-16, 1-14, 1-12, 1-10, 1-8, 1-6, 1-4, 1-2, 2-20, 2-18, 2-6, 2-14, 2-12, 2-10, 2-8, 2-6, 2-4, 4-20, 4-18, 4-16, 4-14, 4-12, 4-10, 4-8, 4-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 12-20, 12-18, 12-16, 12-14, 14-20, 14-18, 14-16, 16-20, 16-18, or 18-20.

In some aspects, the dentifrice, skin care, or cosmetic compositions comprise the amino acid in amount of greater than 0 wt. %. In some aspects, the dentifrice, skin care, or cosmetic compositions comprise the amino acid in amount of 25 wt. % or less. In some aspects, the dentifrice, skin care, or cosmetic compositions comprise the amino acid in amount (wt. %) of 0-25, 0-24, 0-22, 0-20, 0-18, 0-16, 0-14, 0-12, 0-10, 0-8, 0-6, 0-4, 0-2, 0-1, 0-0.5, 0-0.1, >0-25, >0-24, >0-22, >0-20, >0-18, >0-16, >0-14, >0-12, >0-10, >0-8, >0-6, >0-4, >0-2, >0-1, >0-0.5, >0-0.1, 0.1-25, 0.1-24, 0.1-22, 0.1-20, 0.1-18, 0.1-16, 0.1-14, 0.1-12, 0.1-10, 0.1-8, 0.1-6, 0.1-4, 0.1-2, 0.1-1, 0.1-0.5, 0.5-25, 0.5-24, 0.5-22, 0.5-20, 0.5-18, 0.5-16, 0.5-14, 0.5-12, 0.5-10, 0.5-8, 0.5-6, 0.5-4, 0.5-2, 0.5-1, 1-25, 1-24, 1-22, 1-20, 1-18, 1-16, 1-14, 1-12, 1-10, 1-8, 1-6, 1-4, 1-2, 2-25, 2-24, 2-22, 2-20, 2-18, 2-6, 2-14, 2-12, 2-10, 2-8, 2-6, 2-4, 4-25, 4-24, 4-22, 4-20, 4-18, 4-16, 4-14, 4-12, 4-10, 4-8, 4-6, 6-25, 6-24, 6-22, 6-20, 6-18, 6-16, 6-14, 6-12, 6-10, 6-8, 8-25, 8-24, 8-22, 8-20, 8-18, 8-16, 8-14, 8-12, 8-10, 10-25, 10-24, 10-22, 10-20, 10-18, 10-16, 10-14, 10-12, 12-25, 12-24, 12-22, 12-20, 12-18, 12-16, 12-14, 14-25, 14-24, 14-22,14-20, 14-18, 14-16, 16-25, 16-24, 16-22, 16-20, 16-18, 18-25, 18-24, 18-22,18-20, 20-25, 20-24, 20-22, 22-25, 22-24, or 24-25.

In some aspects, the dentifrice, skin care, or cosmetic compositions comprise a solvent in an amount of 10 wt. % or more. In some aspects, the dentifrice, skin care, or cosmetic compositions comprise a solvent in an amount of 80 wt. % or less. In some aspects, the dentifrice, skin care, or cosmetic compositions comprise a solvent in an amount (wt. %) of 10-80, 10-75, 10-70, 10-65, 10-60, 10-55, 10-50,10-45, 10-40, 10-35, 10-30, 10-25, 10-20, 10-15, 15-80, 15-75, 15-70, 15-65, 15-60, 15-55, 15-50, 15-45, 15-40, 15-35, 15-30, 15-25, 15-20, 20-80, 20-75, 20-70, 20-65, 20-60, 20-55, 20-50, 20-45, 20-40, 20-35, 20-30, 20-25, 25-80, 25-75, 25-70, 25-65, 25-60, 25-55, 25-50, 25-45, 25-40, 25-35, 25-30, 30-80, 30-75, 30-70, 30-65, 30-60, 30-55, 30-50, 30-45, 30-40, 30-35, 35-80, 35-75, 35-70, 35-65, 35-60, 35-55, 35-50, 35-45, 35-40, 40-80, 40-75, 40-70, 40-65, 40-60, 40-55, 40-50, 40-45, 45-80, 45-75, 45-70, 45-65, 45-60, 45-55, 45-50, 50-80, 50-75, 50-70, 50-65, 50-60, 50-55, 55-80, 55-75, 55-70, 55-65, 55-60, 60-80, 60-75, 60-70, 60-65, 65-80, 65-75, 65-70, 70-80, 70-75, or 75-80. As disclosed elsewhere herein, the solvent can be aqueous or non-aqueous. Generally, when aqueous, the solvent is or comprises water, and when non-aqueous, the solvent is or comprises glycerin. In some aspects, a combination of water and glycerin is employed, and the weight percent of glycerin in such a mixture can be 1-99, 5-95, 10-90, 15-85, 20-80, 25-75, 30-70, 35-65, 40-60, 45-55, 1-50, 5-45, 10-40, 15-35, 50-95, 55-90, 60-85, or 65-80.

In some aspects, the dentifrice, skin care, or cosmetic compositions comprise one or more humectants. Without wishing to be bound by theory, humectants may be employed to prevent “drying out” of the composition and/or to confer a creamy texture. In some aspects, the humectants can include short-chained polyalcohols For example, short-chained polyalcohols can include glycerol, sorbitol, propylene glycol, polyethylene glycol, or any combination thereof. In some aspects, the dentifrice, skin care, or cosmetic compositions comprise one or more humectants in an amount of 0 wt. % or more than 0 wt. %. In some aspects, the dentifrice, skin care, or cosmetic compositions comprise one or more humectants in an amount of 80 wt. % or less. In some aspects, the dentifrice, skin care, or cosmetic compositions comprise one or more humectants in an amount (wt. %) of 0-80, 0-75, 0-70, 0-65, 0-60, 0-55, 0-50, 0-45, 0-40, 0-35, 0-0, 0-25, 0-20, 0-15, 0-10, 0-5, 0-4, 0-3, 0-2, 0-1, >0-80, >0-75, >0-70, >0-65, >0-60, >0-55, >0-50, >0-45, >0-40, >0-35, >0-30, >0-25, >0-20, >0-15, >0-10, >0-5, >0-4, >0-3, >0-2, >0-1, 1-80, 1-75, 1-70, 1-65, 1-60, 1-55, 1-50, 1-45, 1-40,1-35, 1-30, 1-25, 1-20, 1-15, 1-10, 1-5, 5-80, 5-75, 5-70, 5-65, 5-60, 5-55, 5-50, 5-45, 5-40, 5-35, 5-30, 5-25, 5-20, 5-15, 5-10, 10-80, 10-75, 10-70, 10-65, 10-60, 10-55, 10-50, 10-45, 10-40, 10-35, 10-30, 10-25, 10-20, 10-15, 15-80, 15-75, 15-70, 15-65, 15-60, 15-55, 15-50, 15-45, 15-40, 15-35, 15-30,15-25, 15-20, 20-80, 20-75, 20-70, 20-65, 20-60, 20-55, 20-50, 20-45, 20-40, 20-35, 20-30, 20-25, 25-80, 25-75, 25-70, 25-65, 25-60, 25-55, 25-50, 25-45, 25-40, 25-35, 25-30, 30-80, 30-75, 30-70, 30-65, 30-60, 30-55, 30-50, 30-45, 30-40, 30-35, 35-80, 35-75, 35-70, 35-65, 35-60, 35-55, 35-50, 35-45, 35-40, 40-80, 40-75, 40-70, 40-65, 40-60, 40-55, 40-50, 40-45, 45-80, 45-75, 45-70, 45-65, 45-60, 45-55, 45-50, 50-80, 50-75, 50-70, 50-65, 50-60, 50-55, 55-80, 55-75, 55-70, 55-65, 55-60, 60-80, 60-75, 60-70, 60-65, 65-80, 65-75, 65-70, 70-80, 70-75, or 75-80. In some aspects, the dentifrice, skin care, or cosmetic compositions comprise one or more humectants in an amount (wt. %) of 0-5, 0-4, 0-3, 0-2, 0-1, 0-0.5, 0-0.1, 0-0.01, 0-0.001, 0-0.0001, >0-5, >0-4, >0-3, >0-2, >0-1, >0-0.5, >0-0.1, >0-0.01, >0-0.001, >0-0.0001, 0.0001-5, 0.0001-4, 0.0001-3, 0.0001-2, 0.0001-1, 0.0001-0.5, 0.0001-0.1, 0.0001-0.01, 0.0001-0.001, 0.001-5, 0.001-4, 0.001-3, 0.001-2, 0.001-1, 0.001-0.5, 0.001-0.1, 0.001-0.01, 0.01-5, 0.01-4, 0.01-3, 0.01-2, 0.01-1, 0.01-0.5, 0.01-0.1, 0.1-5, 0.1-4, 0.1-3, 0.1-2, 0.1-1, 0.1-0.5, 0.5-5, 0.5-4, 0.5-3, 0.5-2, 0.5-1, 1-5, 1-4, 1-3, 1-2, 2-5, 2-4, 2-3, 3-5, 3-4, or 4-5. When the dentifrice, skin care, or cosmetic compositions comprise glycerin (also called glycerol) as a solvent, the glycerin may also be acting as a humectant. Generally, therefore, the glycerin does not get double-counted in terms of concentration as both as a solvent and as a humectant.

In some aspects, the dentifrice, skin care, or cosmetic composition comprises one or more fluoride ion sources. In some aspects, the one or more fluoride ion sources include alkali metal fluorides, amine fluorides, stannous fluoride, or any combination thereof. In some aspects, the one or more fluoride ion sources includes calcium fluoride, magnesium fluoride, sodium fluoride, potassium fluoride, stannous fluoride, sodium monofluorophosphate, sodium difluorophosphate, or any combination thereof. In some aspects, the dentifrice, skin care, or cosmetic compositions comprise one or more fluoride ion sources in an amount of 0 wt. % or greater than 0 wt. %. In some aspects, the dentifrice, skin care, or cosmetic compositions comprise one or more fluoride ion sources in an amount of 1 wt. % or less. In some aspects, the dentifrice, skin care, or cosmetic compositions comprise one or more fluoride ion sources in an amount (wt. %) of 0-1, 0-0.9, 0-0.8, 0-0.7, 0-0.6, 0-0.5, 0-0.4, 0-0.3, 0-0.2, 0-0.1, 0-0.05, >0-1, >0-0.9, >0-0.8, >0-0.7, >0-0.6, >0-0.5, >0-0.4, >0-0.3, >0-0.2, >0-0.1, >0-0.05, 0.05-1, 0.05-0.9, 0.05-0.8, 0.05-0.7, 0.05-0.6, 0.05-0.5, 0.05-0.4, 0.05-0.3, 0.05-0.2, 0.05-0.1, 0.1-1, 0.1-0.9, 0.1-0.8, 0.1-0.7, 0.1-0.6, 0.1-0.5, 0.1-0.4, 0.1-0.3, 0.1-0.2, 0.2-1, 0.2-0.9, 0.2-0.8, 0.2-0.7, 0.2-0.6, 0.2-0.5, 0.2-0.4, 0.2-0.3, 0.3-1, 0.3-0.9, 0.3-0.8, 0.3-0.7, 0.3-0.6, 0.3-0.5, 0.3-0.4, 0.4-1, 0.4-0.9, 0.4-0.8, 0.4-0.7, 0.4-0.6, 0.4-0.5, 0.5-1, 0.5-0.9, 0.5-0.8, 0.5-0.7, 0.5-0.6, 0.6-1, 0.6-0.9, 0.6-0.8, 0.6-0.7, 0.7-1, 0.7-0.9, 0.7-0.8, 0.8-1, 0.8-0.9, or 0.9-1.

In some aspects, the dentifrice compositions comprise one or more flavoring agents. In some aspects, the flavoring agents include, for example, essential oils (e.g., peppermint oil, spearmint oil, wintergreen oil, etc., or any combination thereof). In some aspects, the dentifrice compositions comprise one or more flavoring agents in an amount of 0 wt. % or greater than 0 wt. %. In some aspects, the dentifrice compositions comprise one or more flavoring agents in an amount of 0.5 wt. % or less. In some aspects, the dentifrice compositions comprise one or more flavoring agents in an amount (wt. %) of 0-0.5, 0-0.4, 0-0.3, 0-0.2, 0-0.1, 0-0.05, >0-0.5, >0-0.4, >0-0.3, >0-0.2, >0-0.1, >0-0.05, 0.05-0.5, 0.05-0.4, 0.05-0.3, 0.05-0.2, 0.05-0.1, 0.1-0.5, 0.1-0.4, 0.1-0.3, 0.1-0.2, 0.2-0.5, 0.2-0.4, 0.2-0.3, 0.3-0.5, 0.3-0.4, or 0.4-0.5.

In some aspects, the dentifrice, skin care, or cosmetic compositions comprise one or more surfactants. In some aspects, the one or more surfactants include sodium lauryl sulfate, poloxamer (such as an ethylene oxide/propylene oxide copolymers), and any combination thereof. In some aspects, sodium lauryl sulfate exacerbates tooth sensitivity issues and therefore in some aspects sodium lauryl sulfate is not included in the dentifrice compositions. In some aspects, the dentifrice, skin care, or cosmetic compositions comprise one or more surfactants in an amount of 0 wt. % or greater than 0 wt. %. In some aspects, the dentifrice, skin care, or cosmetic compositions comprise one or more surfactants in an amount of 5 wt. % or less. In some aspects, the dentifrice, skin care, or cosmetic compositions comprise one or more surfactants in an amount (wt. %) of 0-5, 0-4.5, 0-4, 0-3.5, 0-3, 0-2.5, 0-2, 0-1.5, 0-1, 0-0.5, 0-0.1, >0-5, >0-4.5, >0-4, >0-3.5, >0-3, >0-2.5, >0-2, >0-1.5, >0-1, >0-0.5, >0-0.1, 0.1-5, 0.1-4.5, 0.1-4, 0.1-3.5, 0.1-3, 0.1-2.5, 0.1-2, 0.1-1.5, 0.1-1, 0.1-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5. In some aspects, the dentifrice, skin care, or cosmetic compositions is substantially free of, or does not include, a surfactant.

In some aspects, the dentifrice compositions comprise one or more sweeteners. In some aspects, the sweeteners include, for example, xylitol, stevia, acesulfame potassium, aspartame, saccharin, sucralose, honey, agave nectar, and the like, or any combination thereof. In some aspects, the dentifrice compositions comprise one or more sweeteners in an amount of 0 wt. % or greater than 0 wt. %. In some aspects, the dentifrice compositions comprise one or more sweeteners in an amount of 5 wt. % or less. In some aspects, the dentifrice compositions comprise one or more surfactants in an amount (wt. %) of 0-5, 0-4.5, 0-4, 0-3.5, 0-3, 0-2.5, 0-2, 0-1.5, 0-1, 0-0.5, 0-0.1, >0-5, >0-4.5, >0-4, >0-3.5, >0-3, >0-2.5, >0-2, >0-1.5, >0-1, >0-0.5, >0-0.1, 0.1-5, 0.1-4.5, 0.1-4, 0.1-3.5, 0.1-3, 0.1-2.5, 0.1-2, 0.1-1.5, 0.1-1, 0.1-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5. In some aspects, the dentifrice composition is substantially free of, or does not include, a sweetener.

In some aspects, the dentifrice, skin care, or cosmetic compositions comprise one or more whitening agents. In some aspects, the one or more whitening agents comprise titanium dioxide, hydrogen peroxide, sodium tripolyphosphates, or any combination thereof. In some aspects, the dentifrice, skin care, or cosmetic compositions comprise one or more whitening agents in an amount of 0 wt. % or greater than 0 wt. %. In some aspects, the dentifrice, skin care, or cosmetic compositions comprise one or more whitening agents in an amount of 2 wt. % or less. In some aspects, the dentifrice, skin care, or cosmetic compositions comprise one or more whitening agents in an amount (wt. %) of 0-2, 0-1.5, 0-1, 0-0.5, 0-0.1, >0-2, >0-1.5, >0-1, >0-0.5, >0-0.1, 0.1-2, 0.1-1.5, 0.1-1, 0.1-0.5, 0.5-2, 0.5-1.5, 0.5-1, 1-2, 1-1.5, or 1.5-2. In some aspects, the dentifrice, skin care, or cosmetic compositions do not contain a whitening agent.

In some aspects, the dentifrice, skin care, or cosmetic compositions comprise one or more abrasives. In some aspects, the one or more abrasives, skin care, or cosmetic comprise silica, zinc orthophosphate, sodium bicarbonate, alumina, calcium carbonate, calcium pyrophosphate, or any combination thereof. Although the bioactive glasses disclosed herein may have an abrasive effect in the dentifrice, skin care, or cosmetic compositions, the bioactive glasses are not considered an abrasive herein for purposes of the amounts or inclusion of the one or more abrasives. In some aspects, the dentifrice, skin care, or cosmetic compositions comprise one or more abrasives in an amount of 0 wt. % or greater than 0 wt. %. In some aspects, the dentifrice, skin care, or cosmetic compositions comprise one or more abrasives in an amount of 20 wt. % or less. In some aspects, the dentifrice, skin care, or cosmetic compositions comprise one or more abrasives in an amount (wt. %) of 0-20, 0-15, 0-10, 0-5, 0-1, 1-20, 1-15, 1-10, 1-5, 5-20, 5-15, 5-10, 10-20, 10-15, or 15-20. In some aspects, the dentifrice, skin care, or cosmetic compositions do not contain an abrasive. In some aspects, the dentifrice, skin care, or cosmetic compositions are substantially free of, or do not contain, one or more of silica, zinc orthophosphate, sodium bicarbonate, alumina, calcium carbonate, calcium pyrophosphate, or any combination thereof.

In some aspects, the dentifrice, skin care, or cosmetic compositions comprise one or more thickening agents (also called gelling agents herein). In some aspects, the one or more thickening agents comprise carboxymethylcellulose (CMC), carrageenan, xanthan gum, a polysaccharide, a synthetic polymer, or any combination thereof. The thickening agents generally are used to modify the rheology/viscosity of the composition and/or to prevent the separation of solid/liquid or liquid/liquid ingredients in the composition. In some aspects, the dentifrice, skin care, or cosmetic compositions comprise one or more thickening agents in an amount of 0 wt. % or greater than 0 wt. %. In some aspects, the dentifrice, skin care, or cosmetic compositions comprise one or more thickening agents in an amount of 15 wt. % or less. In some aspects, the dentifrice, skin care, or cosmetic compositions comprise one or more thickening agents in an amount (wt. %) of 0-15, 0-14, 0-12, 0-10, 0-8, 0-6, 0-4, 0-2, 0-1, 0-0.5, 0-0.1, 0-0.01, >0-15, >0-14, >0-12, >0-10, >0-8, >0-6, >0-4, >0-2, >0-1, >0-0.5, >0-0.1, >0-0.01, 0.01-15, 0.01-14, 0.01-12, 0.01-10, 0.01-8, 0.01-6, 0.01-4, 0.01-2, 0.01-1, 0.01-0.5, 0.01-0.1, 0.1-15, 0.1-14, 0.1-12, 0.1-10, 0.1-8, 0.1-6, 0.1-4, 0.1-2, 0.1-1, 0.1-0.5, 0.5-15, 0.5-14, 0.5-12, 0.5-10, 0.5-8, 0.5-6, 0.5-4, 0.5-2, 0.5-1, 1-15, 1-14, 1-12, 1-10, 1-8, 1-6, 1-4, 1-2, 2-15, 2-14, 2-12, 2-10, 2-8, 2-6, 2-4, 4-15, 4-14, 4-12, 4-10, 4-8, 4-6, 6-15, 6-14, 6-12, 6-10, 6-8, 8-15, 8-14, 8-12, 8-10, 10-15, 10-14, 10-12, 12-15, 12-14, or 14-15. In some aspects, the dentifrice, skin care, or cosmetic compositions do not contain a thickening agent.

In some aspects, the dentifrice, skin care, or cosmetic compositions comprise one or more pH modifiers and/or buffers. In some aspects, the one or more pH modifiers and/or buffers include acids or bases. In some aspects, the acids include organic or inorganic bases, including sulfuric acid, hydrochloric acid, citric acid, oxalic acid, acetic acid, and so forth, or any combination thereof. In some aspects, the bases include organic or inorganic bases, including hydroxides (e.g., sodium hydroxide, potassium hydroxide, lithium hydroxide, ammonium hydroxide, etc.), amine bases (triethyl amine, pyridine, etc.), and so forth, or any combination thereof. In some aspects, buffers include organic or inorganic buffers, including citric acid, acetic acid, KH₂PO₄, Na₂HPO₄, CHES (N-cyclohexyl-2-aminoethanesulfonic acid), borate, and the like, or any combination thereof. In some aspects, the dentifrice, skin care, or cosmetic compositions comprise one or more thickening agents in an amount of 0 wt. % or greater than 0 wt. %. In some aspects, the dentifrice, skin care, or cosmetic compositions comprise one or more thickening agents in an amount of 15 wt. % or less. In some aspects, the dentifrice, skin care, or cosmetic compositions comprise one or more thickening agents in an amount (wt. %) of 0-25, 0-24, 0-22, 0-20, 0-18, 0-16, 0-14, 0-12, 0-10, 0-8, 0-6, 0-4, 0-2, 0-1, 0-0.5, 0-0.1, >0-25, >0-24, >0-22, >0-20, >0-18, >0-16, >0-14, >0-12, >0-10, >0-8, >0-6, >0-4, >0-2, >0-1, >0-0.5, >0-0.1, 0.0001-25, 0.0001-5, 0.0001-1, 0.1-25, 0.1-24, 0.1-22, 0.1-20, 0.1-18, 0.1-16, 0.1-14, 0.1-12, 0.1-10, 0.1-8, 0.1-6, 0.1-4, 0.1-2, 0.1-1, 0.1-0.5, 0.5-25, 0.5-24, 0.5-22, 0.5-20, 0.5-18, 0.5-16, 0.5-14, 0.5-12, 0.5-10, 0.5-8, 0.5-6, 0.5-4, 0.5-2, 0.5-1, 1-25, 1-24, 1-22, 1-20, 1-18, 1-16, 1-14, 1-12, 1-10, 1-8, 1-6, 1-4, 1-2, 2-25, 2-24, 2-22, 2-20, 2-18, 2-6, 2-14, 2-12, 2-10, 2-8, 2-6, 2-4, 4-25, 4-24, 4-22, 4-20, 4-18, 4-16, 4-14, 4-12, 4-10, 4-8, 4-6, 6-25, 6-24, 6-22, 6-20, 6-18, 6-16, 6-14, 6-12, 6-10, 6-8, 8-25, 8-24, 8-22, 8-20, 8-18, 8-16, 8-14, 8-12, 8-10, 10-25, 10-24, 10-22, 10-20, 10-18, 10-16, 10-14, 10-12,12-25, 12-24, 12-22, 12-20, 12-18, 12-16, 12-14, 14-25, 14-24, 14-22, 14-20,14-18, 14-16, 16-25, 16-24, 16-22, 16-20, 16-18, 18-25, 18-24, 18-22, 18-20, 20-25, 20-24, 20-22, 22-25, 22-24, or 24-25. In some aspects, the dentifrice, skin care, or cosmetic compositions do not contain a pH modifier and/or buffer.

In some aspects, the dentifrice, skin care, or cosmetic compositions a pH of 6-10, 6-9.5, 6-9, 6-8.5, 6-8, 6-7.5, 6-7, 6-6.5, 6.5-10, 6.5-9.5, 6.5-9, 6.5-8.5, 6.5-8, 6.5-7.5, 6.5-7, 7-10, 7-9.5, 7-9, 7-8.5, 7-8, 7-7.5, 7.5-10, 7.5-9.5, 7.5-9, 7.5-8.5, 7.5-8, 8-10, 8-9.5, 8-9, 8-8.5, 8.5-10, 8.5-9.5, 8.5-9, 9-10, 9-9.5, or 9.5-10. In some aspects, the dentifrice, skin care, or cosmetic compositions contain a pH modifier and/or buffer in an amount sufficient to achieve such pH values.

In some aspects, the dentifrice, skin care, or cosmetic compositions comprise one or more preservatives. In some aspects, the one or more preservatives comprise sodium benzoate, methylparaben, ethylparaben, or any combination thereof. In some aspects, the dentifrice, skin care, or cosmetic compositions comprise one or more preservatives in an amount of 0 wt. % or greater than 0 wt. %. In some aspects, the dentifrice, skin care, or cosmetic compositions comprise one or more thickening agents in an amount of 0.1 wt. % or less. In some aspects, the dentifrice, skin care, or cosmetic compositions comprise one or more thickening agents in an amount (wt. %) of 0-0.1, 0-0.08, 0-0.06, 0-0.04, 0-0.02, 0-0.015, 0-0.01, 0.001, >0-0.1, >0-0.08, >0-0.06, >0-0.04, >0-0.02, >0-0.015, >0-0.01, >0-0.001, 0.001-0.1, 0.001-0.08, 0.001-0.06, 0.001-0.04, 0.001-0.02, 0.001-0.015, 0.001-0.01, 0.01-1, 0.01-0.08, 0.01-0.06, 0.01-0.04, 0.01-0.02, 0.01-0.015, 0.015-0.1, 0.015-0.08, 0.015-0.06, 0.015-0.04, 0.015-0.02, 0.02-0.1, 0.02-0.08, 0.02-0.06, 0.02-0.04, 0.04-0.1, 0.04-0.08, 0.04-0.06, 0.06-0.1, 0.06-0.08, or 0.08-0.1. In some aspects, the dentifrice, skin care, or cosmetic compositions do not contain a thickening agent.

In some aspects, the dentifrice, skin care, or cosmetic compositions may comprise other ingredients that contribute in some way to a desired dentifrice, skin care, or cosmetic composition. For example, in some aspects, the dentifrice, skin care, or cosmetic compositions can comprise one or more of a colorant, a fragrance, a medication, a preservative, a desensitizing agent (e.g., potassium salts such as potassium nitrate), a pH modifiers, a preservative, a tartar control agent, and so forth, or any combination thereof. In some aspects, the dentifrice, skin care, or cosmetic compositions comprise one or more other ingredients in an amount of 0 wt. % or greater than 0 wt. %. In some aspects, the dentifrice, skin care, or cosmetic compositions comprise one or more other ingredients in an amount of 10 wt. % or less. In some aspects, the dentifrice, skin care, or cosmetic compositions comprise one or more other ingredients in an amount (wt. %) of 0-10, 0-9, 0-8, 0-7, 0-6, 0-5, 0-4, 0-3, 0-2, 0-1, 0-0.5, >0-10, >0-9, >0-8, >0-7, >0-6, >0-5, >0-4, >0-3, >0-2, >0-1, >0-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. The amounts of the one or more ingredients can be used to describe the amount of any one of the other ingredients, or can be used to describe the sum of any two or more of the other ingredients.

In some aspects, the compositions (e.g., dentifrice, skin care, or cosmetic) comprise:
a bioactive glass,
an amino acid,
water, and
one or more of a humectant, a fluoride ion source, a flavoring, a surfactant, a sweetener, a whitening agent, an abrasive, a thickening agent, a pH modifier, a preservative, or any combination thereof.

In some aspects, the compositions (e.g., dentifrice, skin care, or cosmetic) comprise:
a bioactive glass,
an amino acid,
glycerin, and
one or more of a humectant, a fluoride ion source, a flavoring, a surfactant, a sweetener, a whitening agent, an abrasive, a thickening agent, a pH modifier, a preservative, or any combination thereof.

In some aspects, disclosed is a method for making the composition, comprising combining the bioactive glass with a carrier. In some aspects, the carrier is a toothpaste, mouthwash, gel, varnish, rinse, spray, ointment, salve, cream, bandage, polymer film, oral formulation, pill, capsule, or transdermal formulation.

In some aspects, disclosed is a method for making the composition, the method comprising combining the amino acid, the bioactive glass, and optionally one or more of water, glycerin, a humectant, a fluoride ion source, a flavoring, a surfactant, a sweetener, a whitening agent, an abrasive, a thickening agent, a pH modifier, a preservative, or any combination thereof.

In some aspects, disclosed is a method comprising applying the composition to a tooth. In some aspects, the method of applying the composition to a tooth remineralizes the tooth (e.g., the enamel of the tooth). In some aspects, this method is performed by an end user, for example, by way of brushing teeth with a toothpaste, swishing a mouth with a mouthwash, applying dental strips containing the composition, and/or placing a dental tray with a gel or oral formulation into the end user's mouth and in contact with one or more teeth. In some aspects, this method is performed by a professional, such as a doctor, a dentist, or other oral care professional, by way of applying the composition to a patient's teeth, brushing a patient's teeth, directing a patient to swish their mouth with a mouthwash and/or apply dental strips or trays, applying the composition to patient's teeth, and so forth, or any combination thereof.

In some aspects, a dentifrice composition herein may be directly applied to a flexible strip (either by a manufacturing facility or by a user) to form a dental strip, and the dentifrice-containing dental strip then applied to the tooth surface by the user or a dental professional. In some aspects, the formulation may be provided in a separate container (such as a syringe or bottle, such as a squeeze bottle) and then applied to a tray by the user or a dental professional to form a dental tray containing the composition. The dental tray is then worn by the user for a suitable amount of time, for example, to provide a desired level of remineralization. In some aspects, the composition and strips can be provided as a kit, such as flexible strips and a separate container containing the composition so a user can apply the composition to the flexible strips to form dental strips prior to placing the dental strips on one or more teeth. In some aspects, the composition and tray can be provided as a kit, such as a tray or other vessel that generally fits the shape of a user's teeth or portion thereof so a user can add the composition to the tray to form a dental tray prior to placing the dental tray on one or more teeth. In some aspects, the length of application can range from 0.5 hours to 8 hours or more. For example, in some aspects, the length of a single application is for a time period (hours) of 0.5-10, 0.5-9.5, 0.5-9, 0.5-8.5, 0.5-8, 0.5-7.5, 0.5-7, 0.5-6.5, 0.5-6, 0.5-5.5, 0.5-5, 0.5-4.5, 0.5-4, 0.5-3.5, 0.5-3, 0.5-2.5, 0.5-2, 0.5-1.5, 0.5-1, 1-10, 1-9.5, 1-9, 1-8.5, 1-8, 1-7.5, 1-7, 1-6.5, 1-6, 1-5.5, 1-5, 1-4.5, 1-4, 1-3.55, 1-3, 1-2.5, 1-2, 1-1.5, 1.5-10, 1.5-9.5, 1.5-9, 1.5-8.5, 1.5-8, 1.5-7.5, 1.5-7, 1.5-6.5, 1.5-6, 1.5-5.5, 1.5-5, 1.5-4.5, 1.5-4, 1.5-3.5, 1.5-3, 1.5-2.5, 1.5-2, 2-10, 2-9.5, 2-9, 2-8.5, 2-8, 2-7.5, 2-7, 2-6.5, 2-6, 2-5.5, 2-5, 2-4.5, 2-4, 2-3.5, 2-3, 2-2.5, 2.5-10, 2.5-9.5, 2.5-9, 2.5-8.5, 2.5-8, 2.5-7.5, 2.5-7, 2.5-6.5, 2.5-6, 2.5-5.5, 2.5-5, 2.5-4.5, 2.5-4, 2.5-3.5, 2.5-3, 3-10, 3-9.5, 3-9, 3-8.5, 3-8, 3-7.5, 3-7, 3-6.5, 3-6, 3-5.5, 3-5, 3-4.5, 3-4, 3-3.5, 3.5-10, 3.5-9.5, 3.5-9, 3.5-8.5, 3.5-8, 3.5-7.5, 3.5-7, 3.5-6.5, 3.5-6, 3.5-5.5, 3.5-5, 3.5-4.5, 3.5-4, 4-10, 4-9.5, 4-9, 4-8.5, 4-8, 4-7.5, 4-7, 4-6.5, 4-6, 4-5.5, 4-5, 4-4.5, 4.5-10, 4.5-9.5, 4.5-9, 4.5-8.5, 4.5-8, 4.5-7.5, 4.5-7, 4.5-6.5, 4.5-6, 4.5-5.5, 4.5-5, 5-10, 5-9.5, 5-9, 5-8.5, 5-8, 5-7.5, 5-7, 5-6.5, 5-6, 5-5.5, 5.5-10, 5.5-9.5, 5.5-9, 5.5-8.5, 5.5-8, 5.5-7.5, 5.5-7, 5.5-6.5, 5.5-6, 6-10, 6-9.5, 6-9, 6-8.5, 6-8, 6-7.5, 6-7, 6-6.5, 6.5-10, 6.5-9.5, 6.5-9, 6.5-8.5, 6.5-8, 6.5-7.5, 6.5-7, 7-10, 7-9.5, 7-9, 7-8.5, 7-8, 7-7.5, 7.5-10, 7.5-9.5, 7.5-9, 7.5-8.5, 7.5-8, 8-10, 8-9.5, 8-9, 8-8.5, 8.5-10, 8.5-9.5, 8.5-9, 9-10, 9-9.5, or 9.5-10. In some aspects, such an application may be repeated as needed, such as daily, nightly, every other day, every two days, every three days, weekly, every two weeks, every three weeks, every four weeks, monthly, every 2 months, every three months, every six months, once per year, or once every two years. In some aspects, as a result of the rapid mineralization employing the dentifrice compositions disclosed herein, particularly in the form of a dental strip or dental tray, less frequent application of the dentifrice composition can be realized, leading to less cost and less hassle for the user.

In some aspects, the method of applying the composition to a bone remineralizes the bone. In some aspects, the method of applying the composition to a tooth treats caries of the tooth, treats dentin hypersensitivity of the tooth, or any combination thereof. In some aspects, the method of applying the composition to a tooth remineralizes the tooth (e.g., by forming apatite in or on the tooth, such as in or on the enamel of the tooth). In some aspects, the method of applying the composition to a tooth treats caries of the tooth, treats dentin hypersensitivity of the tooth, or any combination thereof, remineralizes the tooth (e.g., enamel) by forming apatite in or on the tooth (e.g., enamel). In some aspects, the method is facilitated by contact with saliva in a mouth (e.g., a patient's mouth).

In some aspects, disclosed is a method comprising applying the composition to skin. In some aspects, this method is performed by an end user, for example, by way of applying the composition as a lotion, salve, etc. to their own skin, such as hands, arms, legs, and/or face. In some aspects, this method is performed by a professional, such as a doctor, a dermatologist, or other skin care professional or pseudo-professional, by way of applying the composition to a patient's skin, directing a patient to apply the composition to their skin, and so forth.

In some aspects, the method treats or reduces the appearance of one or more skin defects, optionally wherein the one or more skin defects comprises wrinkles, blemishes, sagging skin, or any combination thereof.

Various aspects are contemplated herein, several of which are set forth in the paragraphs below. It is explicitly contemplated that any aspect or portion thereof can be combined to form a combination.

Aspect 1: A composition, comprising:
an amino acid; and
a bioactive glass, comprising:
   15-55 wt. % SiO₂;
   0.1-15 wt. % ZrO₂;
   20-60 wt. % CaO; and
   5-30 wt. % P₂O₅;
wherein, when the composition is subjected to artificial saliva of Table 1A or 1B at 37° C., a bioactive crystalline phase is formed within 7 days.

Aspect 2: The composition of aspect 1, or any preceding aspect, wherein, when the composition is subjected to the artificial saliva of Table 1A or 1B at 37° C., the bioactive glass forms a bioactive crystalline phase within 1 day.

Aspect 3: The composition of aspect 1 or 2, or any preceding aspect, wherein the artificial saliva has the composition of Table 1A.

Aspect 4: The composition of any one of aspects 1-3, or any preceding aspect, wherein the artificial saliva has the composition of Table 1B.

Aspect 5: The composition of any one of aspects 1-4, or any preceding aspect, wherein the bioactive crystalline phase comprises apatite, brushite, or a combination thereof.

Aspect 6: The composition of aspect 5, or any preceding aspect, wherein the apatite comprises hydroxyapatite, fluorapatite, carbonated apatite, or any combination thereof.

Aspect 7: The composition of any one of aspects 1-6, or any preceding aspect, wherein the bioactive glass is present in an amount of >0-20 wt. %, based on total weight of the composition.

Aspect 8: The composition of any one of aspects 1-7, or any preceding aspect, wherein the bioactive glass is in a form of particulates, microbeads, or a combination thereof.

Aspect 9: The composition of any one of aspects 1-8, or any preceding aspect, wherein the bioactive glass comprises:
25-35 wt. % SiO₂;
35-50 wt. % CaO; and
15-25 wt. % P₂O₅;
based on total weight of the bioactive glass.

Aspect 10: The composition of any one of aspects 1-9, or any preceding aspect, wherein the bioactive glass comprises: 3-12 wt. % ZrO₂, based on total weight of the bioactive glass.

Aspect 11: The composition of any one of aspects 1-10, or any preceding aspect, wherein the bioactive glass comprises: >0-25 wt. % MgO, based on total weight of the bioactive glass.

Aspect 12: The composition of any one of aspects 1-11, or any preceding aspect, wherein the bioactive glass comprises: >0-5 wt. % F⁻, based on total weight of the bioactive glass.

Aspect 13: The composition of any one of aspects 1-12, or any preceding aspect, wherein the bioactive glass comprises: 0-5 wt. % Na₂O+K₂O+Li₂O, based on total weight of the bioactive glass.

Aspect 14: The composition of any one of aspects 1-13, or any preceding aspect, wherein the bioactive glass comprises: CaO+P₂O₅ in an amount of 40-70 wt. %, based on total weight of the bioactive glass.

Aspect 15: The composition of any one of aspects 1-14, or any preceding aspect, wherein the F in the bioactive glasses is derived from calcium fluoride, magnesium fluoride, sodium fluoride, potassium fluoride, stannous fluoride, sodium monofluorophosphate, sodium difluorophosphate, or any combination thereof.

Aspect 16: The composition of any one of aspects 1-15, or any preceding aspect, wherein: the amino acid is present in an amount of >0-25 wt. %, based on total weight of the composition.

Aspect 17: The composition of any one of aspects 1-16, or any preceding aspect, wherein the amino acid is present in an amount of 5-15 wt. %, based on total weight of the composition.

Aspect 18: The composition of any one of aspects 1-17, or any preceding aspect, wherein the amino acid is glycine, glutamic acid, or a combination thereof.

Aspect 19: The composition of any one of aspects 1-18, or any preceding aspect, wherein the amino acid is histidine, lysine, pyrrolysine, aspartic acid, serine, threonine, asparagine, glutamine, cysteine, selenocysteine, proline, alanine, valine, isoleucine, leucine, methionine, phenylalanine, tyrosine, tryptophan, or any combination thereof.

Aspect 20: The composition of any one of aspects 1-19, or any preceding aspect, wherein the amino acid is arginine.

Aspect 21: The composition of any one of aspects 1-19, or any preceding aspect, wherein the amino acid is not arginine.

Aspect 22: The composition of any one of aspects 1-21, or any preceding aspect, wherein the composition is substantially free of, or does not contain calcium carbonate.

Aspect 23: The composition of any one of aspects 1-22, or any preceding aspect, wherein: the composition is in a form of a toothpaste, mouthwash, gel, varnish, rinse, spray, ointment, salve, cream, bandage, polymer film, oral formulation, pill, capsule, transdermal formulation, or any combination thereof.

Aspect 24: The composition of any one of aspects 1-23, or any preceding aspect, wherein the composition is a dentifrice composition.

Aspect 25: The composition of any one of aspects 1-23, or any preceding aspect, wherein the composition is a skin care composition.

Aspect 26: The composition of any one of aspects 1-25, or any preceding aspect, wherein the composition comprises:
water, and
one or more of a humectant, a fluoride ion source, a flavoring, a surfactant, a sweetener, a whitening agent, an abrasive, a thickening agent, a pH modifier, a preservative, or any combination thereof.

Aspect 27: The composition of any one of aspects 1-25, or any preceding aspect, wherein the composition comprises:
glycerin, and
one or more of a humectant, a fluoride ion source, a flavoring, a surfactant, a sweetener, a whitening agent, an abrasive, a thickening agent, a pH modifier, a preservative, or any combination thereof.

Aspect 28: A method for making the composition of any one of aspects 1-27, or any preceding aspect, comprising combining the amino acid, the bioactive glass, and optionally one or more of water, glycerin, a humectant, a fluoride ion source, a flavoring, a surfactant, a sweetener, a whitening agent, an abrasive, a thickening agent, a pH modifier, a preservative, or any combination thereof.

Aspect 29: A method comprising applying to a tooth the composition of any one of aspects 1-27, or any preceding aspect.

Aspect 30: The method of aspect 29, or any preceding aspect, wherein the method remineralizes enamel of the tooth, treats caries of the tooth, treats dentin hypersensitivity of the tooth, or any combination thereof.

Aspect 31: The method of aspect 29 or aspect 30, wherein a dental strip or dental tray comprises the composition.

Aspect 32: A method comprising applying to skin the composition of any one of aspects 1-27, or any preceding aspect.

Aspect 33: The method of aspect 32, or any preceding aspect, wherein the method treats or reduces the appearance of one or more skin defects, optionally wherein the one or more skin defects comprises wrinkles, blemishes, sagging skin, or any combination thereof.

Aspect 34: A method for making the bioactive glass of the composition of any one of aspects 1-27, or any preceding aspect, comprising:
combining each component of the bioactive glass to form a mixture,
heating the mixture to a temperature of 1500° C. or less to form a melted mixture, and
cooling the melted mixture to form a cooled mixture.

Aspect 35: A dental strip comprising the composition of any one of aspects 1-27.

Aspect 36: A dental tray comprising the composition of any one of aspects 1-27.

Aspect 37: A kit comprising flexible strips and a container comprising the composition of any one of aspects 1-27.

Aspect 38: A kit comprising a tray and a container comprising the composition of any one of aspects 1-27.

Aspect 39: The dental strip, dental tray, or kit of any one of aspects 35-38, wherein the composition further comprises one or more of water, glycerin, a humectant, a fluoride ion source, a flavoring, a surfactant, a sweetener, a whitening agent, an abrasive, a thickening agent, a pH modifier, a preservative, or any combination thereof.

Aspect 40: A combination of any two or more of aspects 1-33, or any one or more portions thereof.

### EXAMPLES

The following examples illustrate non-limiting aspects of the disclosure and are not intended to be limiting on the scope of the disclosure or claims.

### Example 1—Bioactive Glasses and Immersion Experiments

Non-limiting examples of amounts of precursor oxides for forming bioactive glasses are listed in Table 3.

TABLE 3

| Oxide (wt. %) | 45S5 | 1 | 2 | 3 | 4 | 5 |
|---|---|---|---|---|---|---|
| SiO₂ | 45.0 | 29.5 | 39.5 | 39.5 | 39.5 | 39.5 |
| Na₂O | 24.5 | 0.0 | 0.0 | 0.0 | 0.0 | 0.0 |
| MgO | 0.0 | 4.5 | 4.5 | 9.5 | 14.5 | 0 |
| CaO | 24.5 | 42.5 | 42.5 | 37.5 | 32.5 | 32.5 |
| SrO | 0.0 | 0.0 | 0 | 0 | 0 | 14.5 |
| ZnO | 0.0 | 0.0 | 0 | 0 | 0 | 0 |
| P₂O₅ | 6.0 | 19.5 | 9.5 | 9.5 | 9.5 | 9.5 |
| ZrO₂ | 0.0 | 4.0 | 4 | 4 | 4 | 4 |
| Density* | 2.71 | 3.03 | 2.96 | 2.95 | 2.99 | 3.18 |

| Oxide (wt. %) | 6 | 7 | 8 | 9 | 10 | 11 | 12 |
|---|---|---|---|---|---|---|---|
| SiO₂ | 39.5 | 37.5 | 34.5 | 29.5 | 24.5 | 24.5 | 20.5 |
| Na₂O | 0.0 | 0.0 | 0.0 | 0.0 | 0.0 | 0.0 | 0.0 |
| MgO | 0 | 4.5 | 4.5 | 4.5 | 4.5 | 4.5 | 4.5 |
| CaO | 32.5 | 42.5 | 42.5 | 42.5 | 42.5 | 42.5 | 42.5 |
| SrO | 0 | 0.0 | 0.0 | 0.0 | 0.0 | 0.0 | 0.0 |
| ZnO | 14.5 | 0.0 | 0.0 | 0.0 | 0.0 | 0.0 | 0.0 |
| P₂O₅ | 9.5 | 11.5 | 14.5 | 19.5 | 24.5 | 22.5 | 22.5 |
| ZrO₂ | 4 | 4.0 | 4.0 | 4.0 | 4.0 | 6 | 10 |
| Density* | 3.15 | 2.98 | 2.99 | 3.03 | 3.12 | 3.16 | 3.26 |

*g/cm³

The bioactive glasses of Table 3 were prepared by conventional melting and forming techniques. Generally, the compositions may be melted at temperatures below 1500° C., or at temperatures below 1400° C., or at temperatures below 1200° C., thereby making it possible to melt in relatively small commercial glass tanks. The bioactive glasses disclosed herein (e.g., Table 3) exhibit significantly higher chemical durability and excellent bioactivity, as compared to currently available glasses, and can be in any form that is useful for the medical, dental, and/or cosmetic processes disclosed. In some aspects, Sample 1 of Table 3 demonstrates significantly higher chemical durability and bioactivity over the 45S5 glass, which is employed as a comparative example herein.

Table 4 below includes example concentrations of amino acids used in the artificial saliva submersion experiments with bioactive glass.

TABLE 4

| Amino Acid | wt. % |
|---|---|
| Glycine | 0-25 |
| L-Glutamic Acid | 0-8 |
| L-Arginine | 0-8 |

Table 5 below includes artificial saliva composition and submersion conditions for the bioactive glass and amino acids.

TABLE 5

| Artificial Saliva | | | | |
|---|---|---|---|---|
| Composition | wt. % | Experiment Details | | |
| Potassium chloride | 0.119 | Temperature | 37° | C. |
| Ammonium chloride | 0.024 | Glass Mass | 5.0 | g |
| Potassium dihydrogen | 0.054 | Amino acid mass | 0-25.0 | g |
| phosphate | | | | |
| Magnesium chloride | 0.004 | Solution Volume | 100 | mL |
| hexahydrate | | | | |

| | | | |
|---|---|---|---|
| Calcium chloride | 0.011 | pH | 6.8 |
| HEPES buffer | 0.477 | Time | 1 hour to 7 days |
| Sodium azide | 0.020 | Powder | <10 μm particles |
| Sodium hydroxide | 0.012 | | |

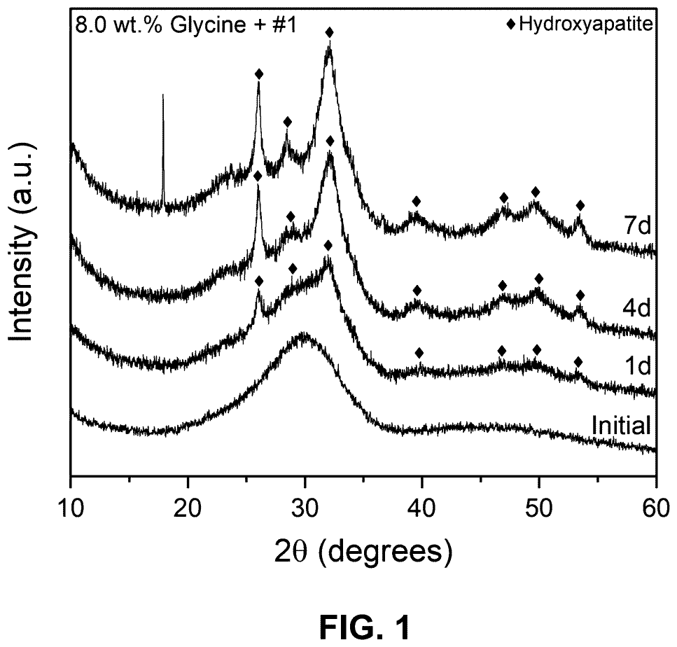

Immersion experiments were performed on the two glasses set forth in Table 3, employing the amino acid and artificial saliva concentrations and immersion conditions set forth in Tables 4 and 5. The results are set forth in FIGS. 1-4 and FIGS. 9-11.

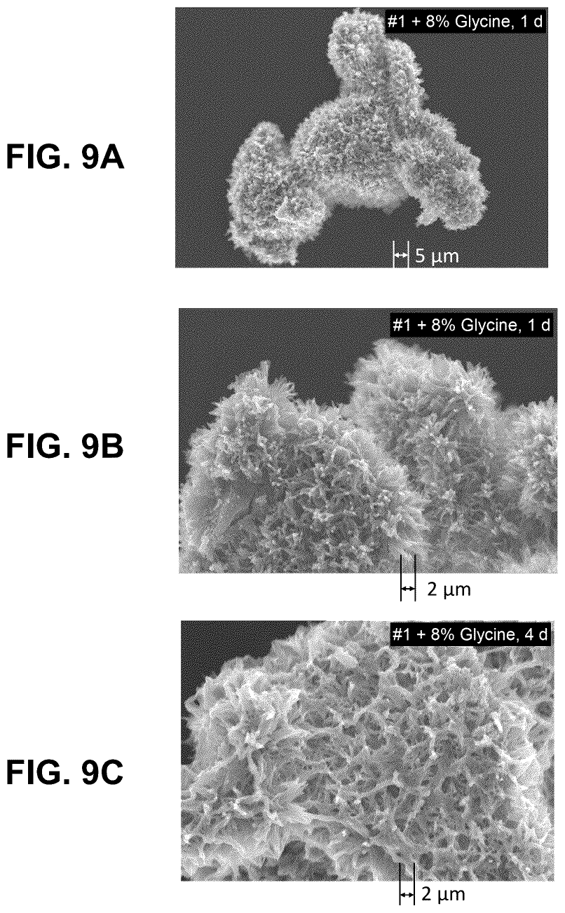

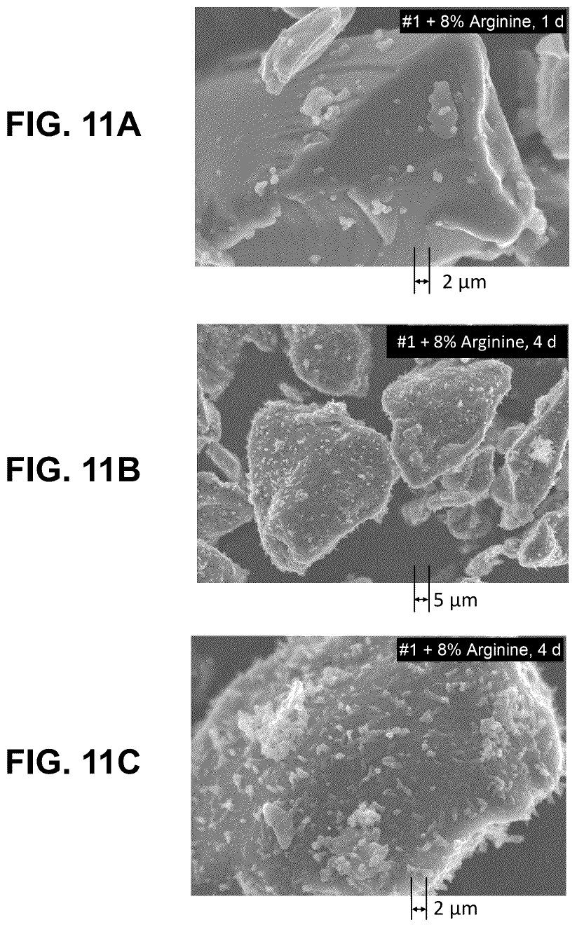

FIG. 1 is a powder XRD spectrum illustrating hydroxyapatite formation within 1 day for bioactive glass 1 of Table 3 upon immersion in an aqueous composition comprising the artificial saliva of Table 5 and an amino acid (8 wt. % glycine). The XRD peaks for hydroxyapatite grow as the submersion time increases from 1 day, to 4 days, to 7 days. FIGS. 9A, 9B, and 9C depict scanning electron microscopy (SEM) images of bioactive glass 1 of Table 3 after immersion in artificial saliva comprising 8 wt. % glycine after 1 day (FIGS. 9A and 9B) and after 4 days (FIG. 9C). The SEM parameters are as follows: FIG. 9A: 5.0 kV 6.2 mm×10.0 k SE(M), scale marks 5.00 microns; FIG. 9B: 5.0 kV 6.2 mm×25.0 k SE(M), scale marks 2.00 microns; FIG. 9C: 5.0 kV 6.4 mm×25.0 k SE(M), scale marks 2.00 microns. Compared with FIGS. 11A, 11B, and 11C (arginine), significantly more crystal growth is observed on the surface of bioactive glass 1 when employing glycine (FIGS. 9A, 9B, and 9C). However, in some aspects, it may be desirable to employ arginine, alone or in combination with other amino acids, so as to tune the amount and type of crystal growth.

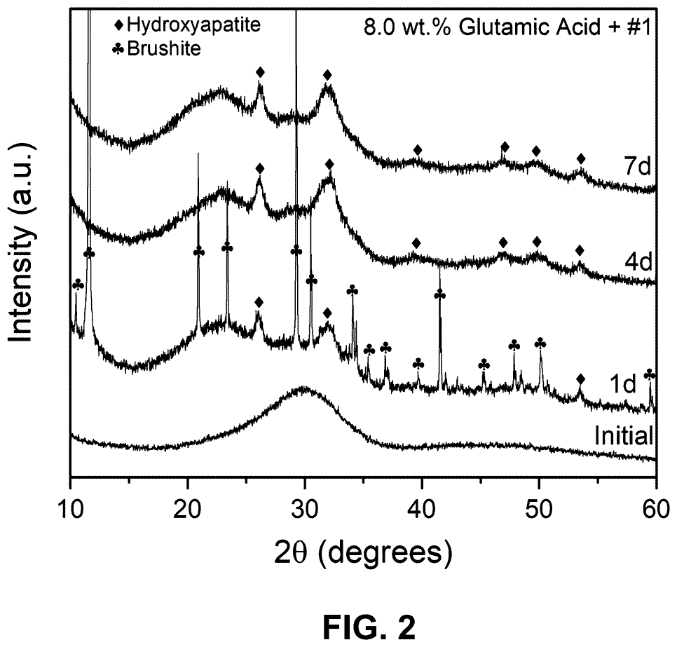

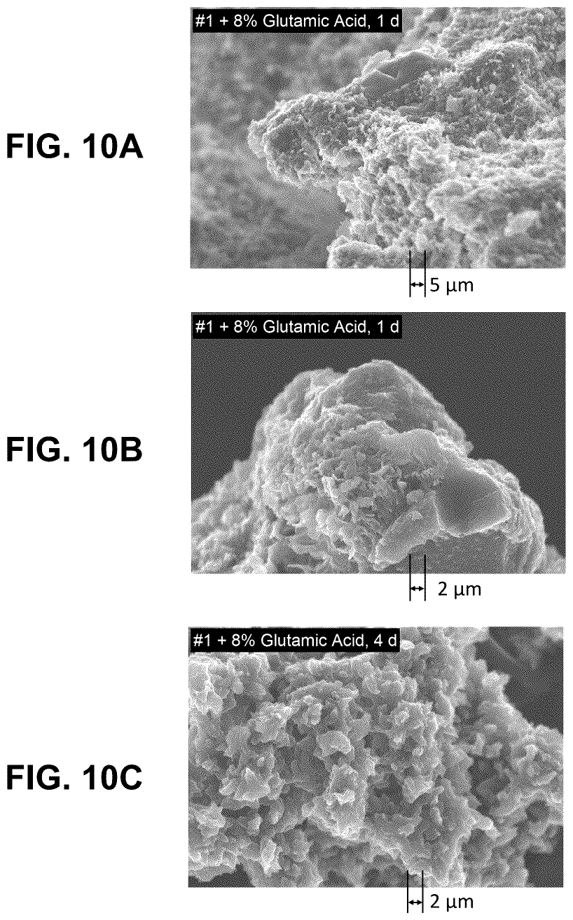

FIG. 2 is a powder XRD spectrum illustrating hydroxyapatite and brushite formation within one day for bioactive glass 1 of Table 3 upon immersion in an aqueous composition comprising the artificial saliva of Table 5 and an amino acid (8 wt. % glutamic acid). The XRD peaks for hydroxyapatite grow as the submersion time increases from 1 day, to 4 days, to 7 days. Brushite forms with bioactive glass 1 within 1 day and then disappears as the submission time increases up to 7 days. Brushite is a potential precursor to hydroxyapatite, and brushite may initially form but then convert substantially or completely to hydroxyapatite over time. FIGS. 10A, 10B, and 10C depict SEM images of bioactive glass 1 of Table 3 after immersion in artificial saliva comprising 8 wt. % glutamic acid after 1 day (FIGS. 10A and 10B) and after 4 days (panel C). The SEM parameters are as follows: FIG. 10A: 5.0 kV 6.2 mm×10.0 k SE(M), scale marks 5.00 microns; FIG. 10B: 5.0 kV 6.3 mm×25.0 k SE(M), scale marks 2.00 microns; FIG. 10C: 5.0 kV 6.4 mm×25.0 k SE(M), scale marks 2.00 microns. Compared with FIGS. 11A, 11B, and 11C (arginine), significantly more crystal growth is observed on the surface of bioactive glass 1 when employing glutamic acid (FIGS. 10A, 10B, and 10C). However, in some aspects, it may be desirable to employ arginine, alone or in combination with other amino acids, so as to tune the amount and type of crystal growth.

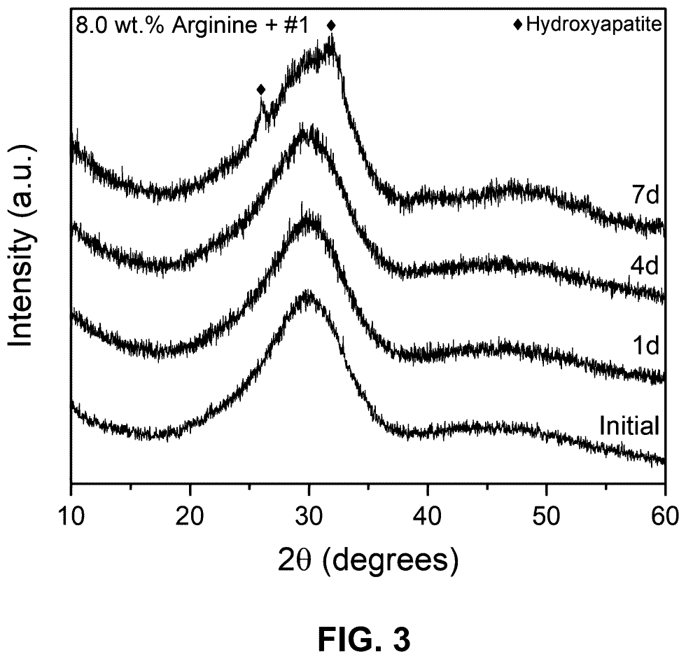

FIG. 3 is a powder XRD spectrum illustrating hydroxyapatite formation at 7 days for bioactive glass 1 of Table 3 upon immersion in an aqueous composition comprising the artificial saliva of Table 5 and an amino acid (8 wt. % arginine). FIGS. 11A, 11B, and 11C depict SEM images of bioactive glass 1 of Table 3 after immersion in artificial saliva comprising 8 wt. % arginine after 1 day (FIG. 11A) and after 4 days (FIGS. 11B and 11C). The SEM parameters are as follows: FIG. FIG. 11A: 5.0 kV 6.4 mm×25.0 k SE(M), scale marks 2.00 microns; 11B: 5.0 kV 6.3 mm×10.0 k SE(M), scale marks 5.00 microns; FIG. 11C: 5.0 kV 6.3 mm×25.0 k SE(M), scale marks 2.00 microns. Compared with FIGS. 9A, 9B, and 9C (glycine) and FIGS. 10A, 10B, and 10C (glutamic acid), significantly less crystal growth is observed on the surface of bioactive glass 1 when employing arginine (FIGS. 11A, 11B, and 11C). However, in some aspects, it may be desirable to employ arginine, alone or in combination with other amino acids, so as to tune the amount and type of crystal growth.

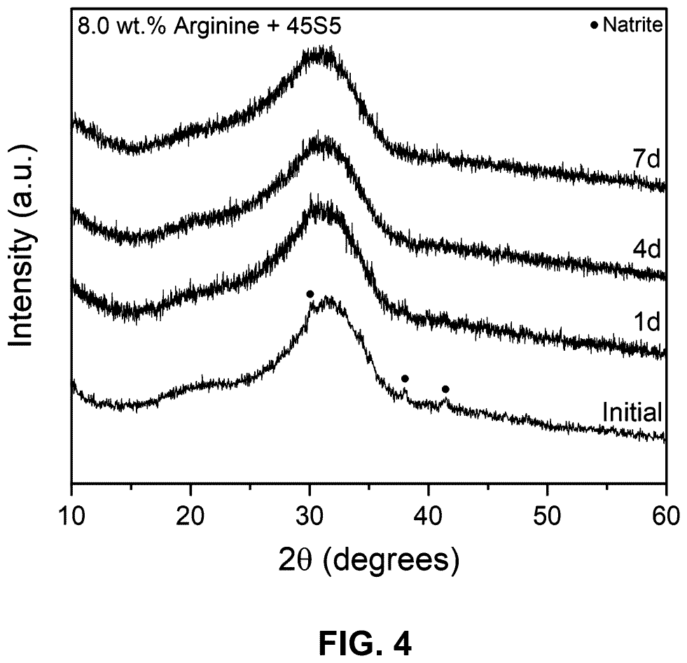

FIG. 4 is a powder XRD spectrum illustrating no mineral phase formation, including no hydroxyapatite formation, within 7 days for the 45S5 Bioglass® comparative glass composition upon immersion in an aqueous composition comprising the artificial saliva of Table 5 and 8 wt. % arginine. As specified by ASTM F1538-03, bioactive materials only refer to those that can form apatite in vitro or in vivo. It is believed that the formation of natrite would not produce a direct bond with bony tissues due to its chemical difference from bone; thus, natrite is not regarded as bioactive. The inclusion of relatively high concentrations of CaO and P₂O₅ oxides, as well as the believed nucleating and crystal growth promoting ability of amino acids, for the compositions disclosed herein facilitates the formation of an apatite phase in artificial saliva while the 45S5 comparative glass composition (which has appreciably lower concentrations of CaO and P₂O₅) only forms a natrite phase in an aqueous composition comprising arginine. Due to its chemical similarity with bone, apatite can form a direct bond with bony tissue while natrite cannot.

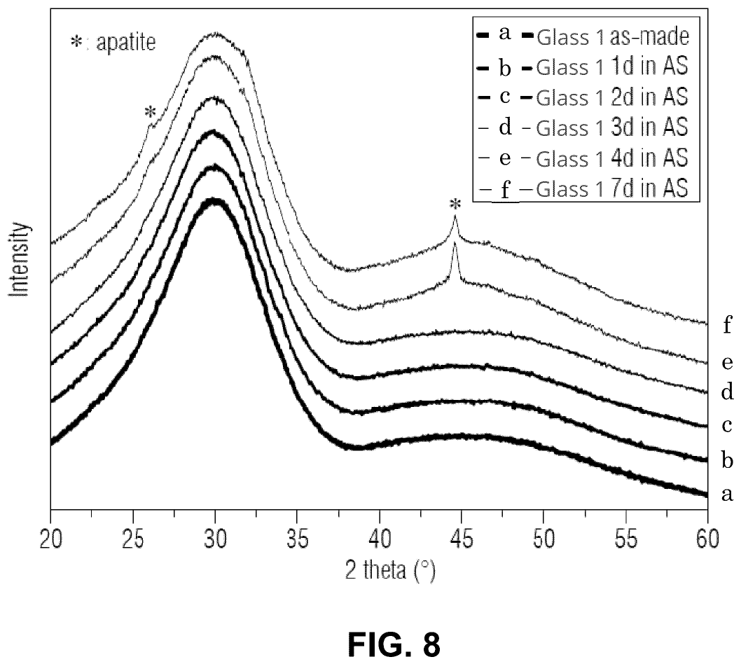

For comparison purposes, bioactive glass 1 was immersed in an artificial saliva manufactured by Modus Laboratories in the absence of any added amino acid. The results are shown in FIG. 8. Specifically, six XRD plots were collected, including bioactive glass 1 in its as-made form, and then after immersion for 1 day, after 2 days, after 3 days, after 4 days, and after 7 days in the artificial saliva. The XRD plots in FIG. 8 show that apatite formation occurred on the bioactive glass within 4 days of soaking in artificial saliva without amino acid present, and significant additional formation of apatite did not occur between days 4 and 7. This shows that inclusion of amino acid in the artificial saliva results in much more rapid apatite formation (within 1 day) than when amino acid is omitted (within 4 days).

### Example 2—pH as a Function of Immersion Time

The two bioactive glasses of Table 3 were immersed in the artificial saliva of Table 5, along with 8 wt. % of specific amino acids (glycine, glutamic acid, or arginine), and the pH monitored over 7 days. The pH of the artificial saliva of Table 5 was 6.8.

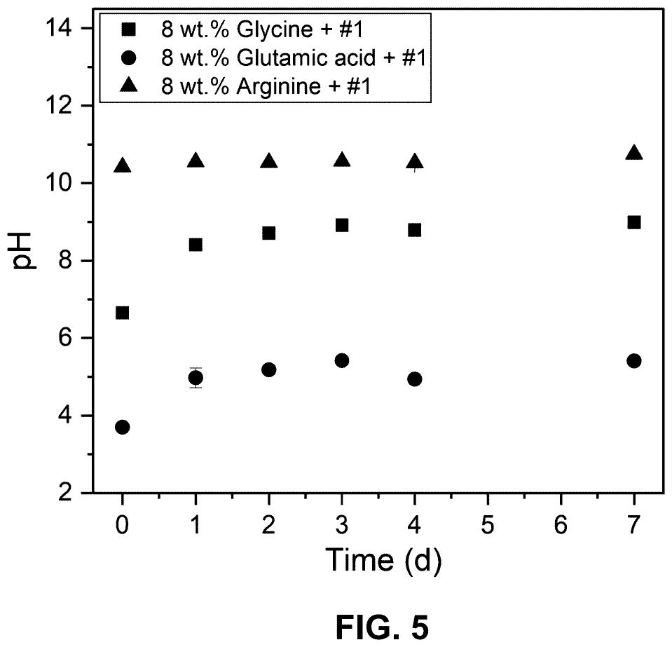

FIG. 5 is a graph illustrating pH change over time upon separately immersing bioactive glass 1 of Table 3 in an aqueous composition comprising the artificial saliva of Table 5 and three different amino acids (glycine, glutamic acid, and arginine). As illustrated in FIG. 5, the pH increases over time, which, without wishing to be bound by theory, is believed to result from dissolution of the glass over time.

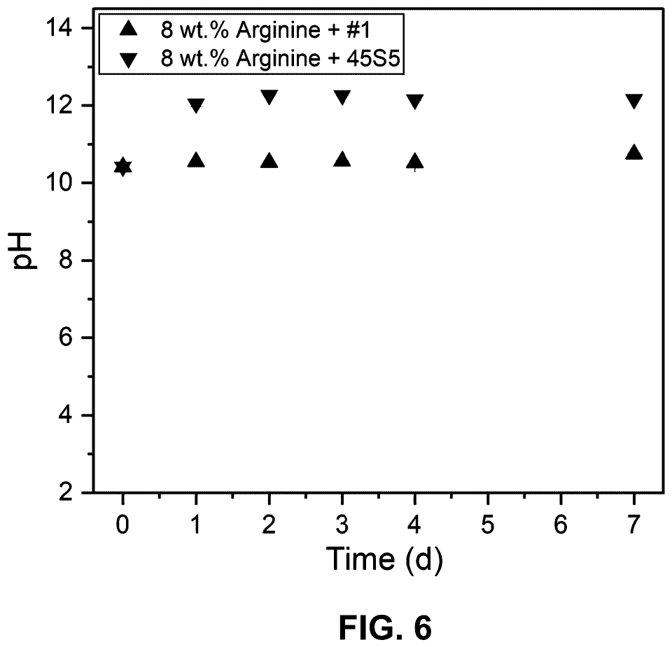

In contrast, FIG. 6 compares the pH over time for bioactive glass 1 of Table 3 with the pH over time for 45S5 Bioglass®, where both glasses are immersed in an aqueous composition comprising the artificial saliva of Table 5 in combination with 8 wt. % arginine. As shown in FIG. 6, the 45S5 glass reached higher pH values, possibly due to relatively greater dissolution of the 45S5 glass compared to bioactive glass 1.

### Example 3—45S5 Without Amino Acid

This example demonstrates that 45S5 Bioglass® fails to form a crystalline phase when immersing in artificial saliva for 7 days without any amino acid present.

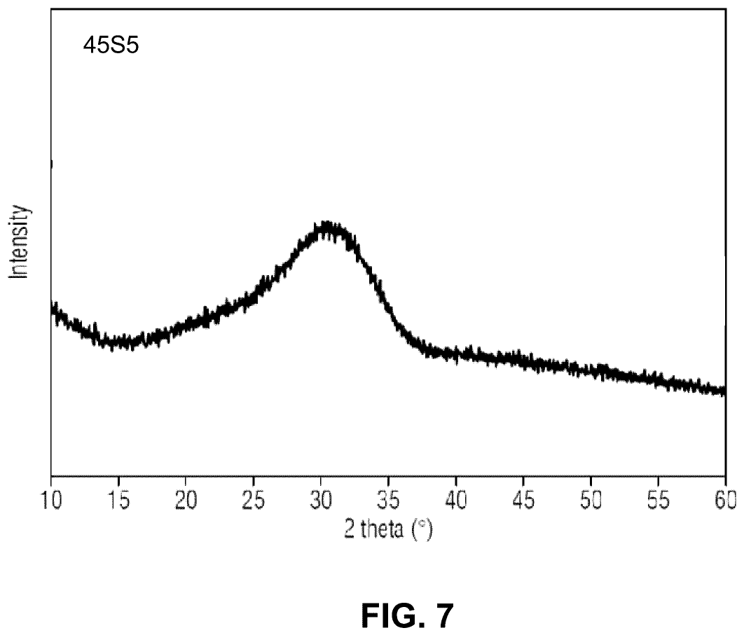

45S5 Bioglass® particles were soaked in artificial saliva manufactured by Modus Laboratories, without any amino acid present. XRD analysis was then performed on the particles. The XRD result in FIG. 7 show no detectable crystalline phase was observed in 45S5 Bioglass® after 7 days. Again, as discussed above, as specified by ASTM F1538-03, bioactive materials only refer to those that can form apatite in vitro or in vivo. In this context for 45S5 Bioglass®, without the formation of apatite, a direct bond with bony tissues would not result.

### Example 4—Rapid Mineralization of Human Enamel Discs

This example demonstrates the ability of the bioactive glasses disclosed herein to rapidly mineralize (e.g., within 1 or 4 days) human enamel discs in artificial saliva solutions with or without amino acid, as compared to 45S5 Bioglass® or CaCO₃.

In this example, 45S5 Bioglass® and the bioactive glass of Composition 1 in Table 3 were subjected to artificial saliva immersion experiments according to the details of Table 5, except that human enamel discs were also present in the artificial saliva composition. The human enamel discs were supplied as extracted human tooth samples commercially available from Intertek (UK). The samples were enamel blocks cut from premolars, sized approximately 4.2 mm×4.2 mm×3 mm, embedded in acrylic resin. The enamel discs were ground with an abrasive disc to flatten the surface and then polished to a final finish of 0.3 microns with a polishing agent.

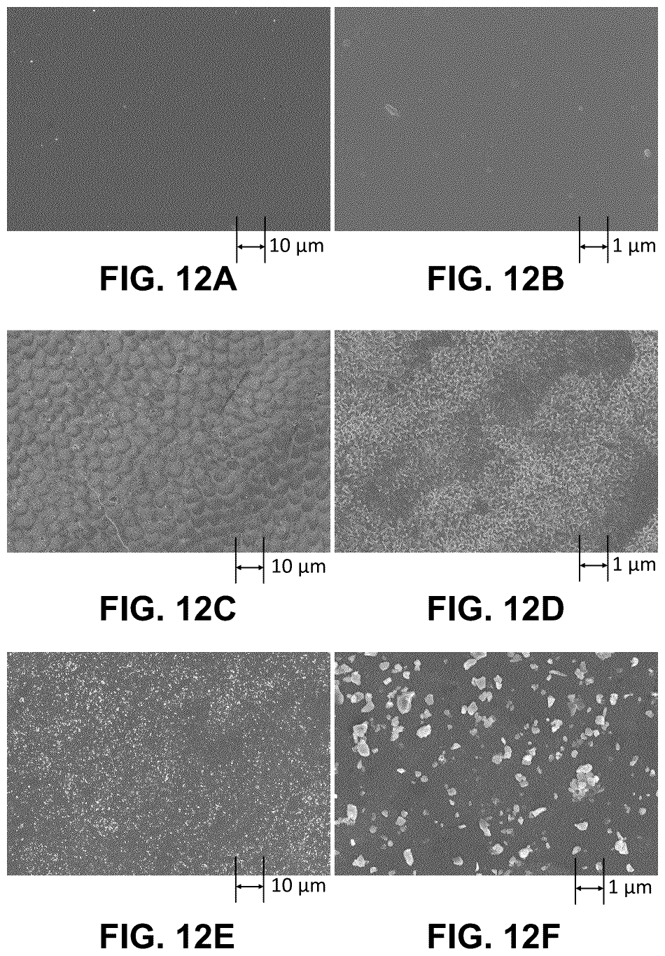

Specifically, different human enamel discs were immersed in Table 5's artificial saliva composition for 4 days at 37° C. with or without certain components. One sample was used as a control (i.e., with no other components besides the human enamel discs in the artificial saliva), with SEM results shown in FIGS. 12A and 12B, which are different magnifications of the same sample. A different human enamel disc was immersed at the same time/temperature conditions but with 5 wt. % of the Composition 1 bioactive glass of Table 3. SEM results are shown in FIGS. 12C and 12D, which are different magnifications of the same sample. A third human enamel disc was immersed at the same time/temperature conditions but with 5 wt. % CaCO₃. SEM results are shown in FIGS. 12E and 12F, which are different magnifications of the same sample. None of these artificial saliva compositions contained amino acid. As illustrated by FIGS. 12A-12D, remineralization (hydroxyapatite formation) of tooth enamel can be achieved by immersing it for 4 days in an artificial saliva that contains 5 wt % of glass powder. A typical “keyhole” morphology composed of nano-sized mineralized hydroxyapatite crystals can be observed in the human enamel disc treated with Composition 1 bioactive glass in Table 3. In comparison, no hydroxyapatite remineralization was observed in human enamel discs immersed in an artificial saliva without bioactive glass. The presence of CaCO₃ in artificial saliva did not promote hydroxyapatite remineralization either, as confirmed by SEM images.

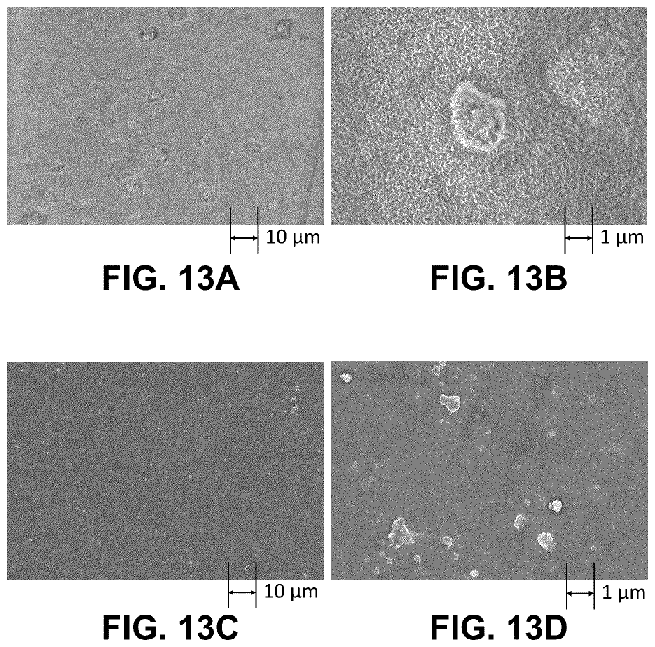

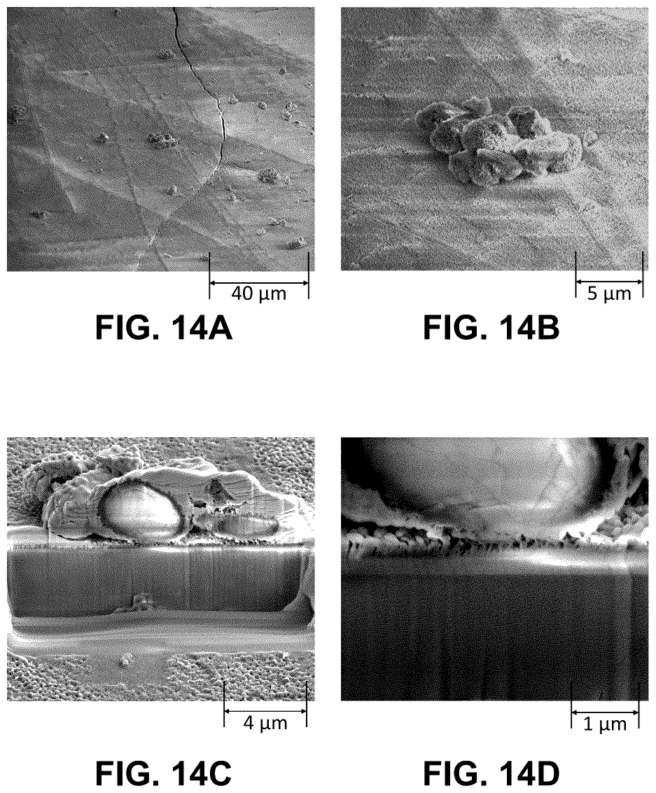

Experiments were also performed with human enamel discs where the artificial saliva composition contains amino acid along with either the Composition 1 bioactive glass of Table 3 or as a comparative example 45S5 Bioglass®. Specifically, a human enamel disc was immersed at 37° C. for 1 day in Table 5's artificial saliva composition along with 5 wt. % of Composition 1 of Table 3 and 8 wt. % of glycine, according to the details of the immersion experiments of Table 5. SEM results are shown in FIGS. 13A and 13B, which are different magnifications of the same sample, as well as in FIGS. 14A-14D. The samples of FIGS. 14A-14D were milled using a focused ion beam (FIB). In addition, a human enamel disc was immersed at 37° C. for 4 days in Table 5's artificial saliva composition along with 45S5 Bioglass® and 8 wt. % glycine. SEM results are shown in FIGS. 13C and 13D, which are different magnifications of the same sample. As illustrated in FIGS. 13A-13D and 14A-14D, a faster hydroxyapatite remineralization within 1 day can be obtained when a combination of Composition 1 bioactive glass from Table 3 and glycine in artificial saliva is used. Without wishing to be bound by theory, it is believed that glycine can promote the nucleation and/or growth of apatite crystals on enamel discs for efficient remineralization. In comparison, remineralization of enamel discs immersed in artificial saliva containing 45S5 Bioglass® and glycine was not observed, even after 4 days. This strongly indicates the importance of the glass composition on its remineralization ability. Moreover, FIGS. 14A-14D illustrate that a strong interfacial bonding can be formed between the bioactive glass and the enamel surface. In particular, the FIB cross-sectional images show chemical bonding through the remineralization process by an apatite layer. This type of bonding is understood in the field to be an indication of high bioactivity.

As shown by these experiments, remineralization in human enamel discs occurs within 4 days when the human enamel discs are immersed in an artificial saliva composition containing a bioactive glass composition in accordance with the disclosure (e.g., Composition 1 of Table 3), or within 1 day when immersed in an artificial saliva composition containing a combination of a bioactive glass in accordance with the disclosure and an amino acid (e.g., glycine). In comparison, no sign of remineralization of the human enamel discs was observed after 4 days in CaCO₃-containing artificial saliva or in artificial saliva containing 45S5 Bioglass® and glycine. Such results indicate, among other things, that the rate of mineralization can be adjusted as desired by tuning the amount of amino acid in a system that employs bioactive glasses in accordance with this disclosure (see also, e.g., Example 5 that compares 8 wt. % amino acid with 25 wt. % amino acid). Without wishing to be bound by theory, it is believed that the Composition 1 bioactive glass provides calcium and phosphate ions for hydroxyapatite formation, whereas the amino acid glycine promotes rapid mineralization perhaps via a biomimetic pathway influencing the rate of crystal nucleation and/or growth.

The SEM parameters for FIGS. 12-14 are as follows: FIG. 12A: CS Gemini 500; WD=6.5 mm; EHT=5.00 kV; Mag=1.00 K X; Signal A=InLens; high vacuum; scale marks 10 microns; FIG. 12B: CS Gemini 500; WD=6.0 mm; EHT=5.00 kV; Mag=10.00 K X; Signal A=InLens; high vacuum; scale marks 1 micron; FIG. 12C: CS Gemini 500; WD=6.1 mm; EHT=5.00 kV; Mag=1.00 K X; Signal A=InLens; high vacuum; scale marks 10 microns; FIG. 12D: CS Gemini 500; WD=6.1 mm; EHT=5.00 kV; Mag=10.00 K X; Signal A=InLens; high vacuum; scale marks 1 micron; FIG. 12E: CS Gemini 500; WD=6.0 mm; EHT=5.00 kV; Mag=1.00 K X; Signal A=InLens; high vacuum; scale marks 10 microns; FIG. 12F: CS Gemini 500; WD=6.0 mm; EHT=5.00 kV; Mag=10.00 K X; Signal A=InLens; high vacuum; scale marks 1 micron; FIG. 13A: CS Gemini 500; WD=6.1 mm; EHT=5.00 kV; Mag=1.00 K X; Signal A=InLens; high vacuum; scale marks 10 microns; FIG. 13B: CS Gemini 500; WD=6.1 mm; EHT=5.00 kV; Mag=10.00 K X; Signal A=InLens; high vacuum; scale marks 1 micron; FIG. 13C: CS Gemini 500; WD=6.4 mm; EHT=5.00 kV; Mag=1.00 K X; Signal A=InLens; high vacuum; scale marks 10 microns; FIG. 13D: CS Gemini 500; WD=6.4 mm; EHT=5.00 kV; Mag=10.00 K X; Signal A=InLens; high vacuum; scale marks 1 micron; FIG. 14A: HV 5.00 kV; curr 26.6 pA; WD 9.8 mm; mag 1200×; tilt 52°; scale marks 40 microns; FIG. 14B: HV 5.00 kV; curr 26.6 pA; WD 9.8 mm; mag 6500×; tilt 52°; scale marks 5 microns; FIG. 14C: HV 5.00 kV; curr 26.6 pA; WD 9.8 mm; mag 10,000×; tilt 52°; scale marks 4 microns; FIG. 14A: HV 5.00 kV; curr 26.6 pA; WD 9.8 mm; mag 65,000×; tilt 52°; scale marks 1 micron.

### Example 5—Rapid Mineralization of Bioactive Glass

This example demonstrates the ability of the bioactive glasses disclosed herein to rapidly mineralize (e.g., within 4 hours) when immersed in artificial saliva solutions with amino acid, as compared to 45S5 Bioglass® or CaCO₃ when immersed in the same artificial saliva solutions with amino acid. This example also demonstrates that rate of mineralization can be affected by concentration of amino acid.

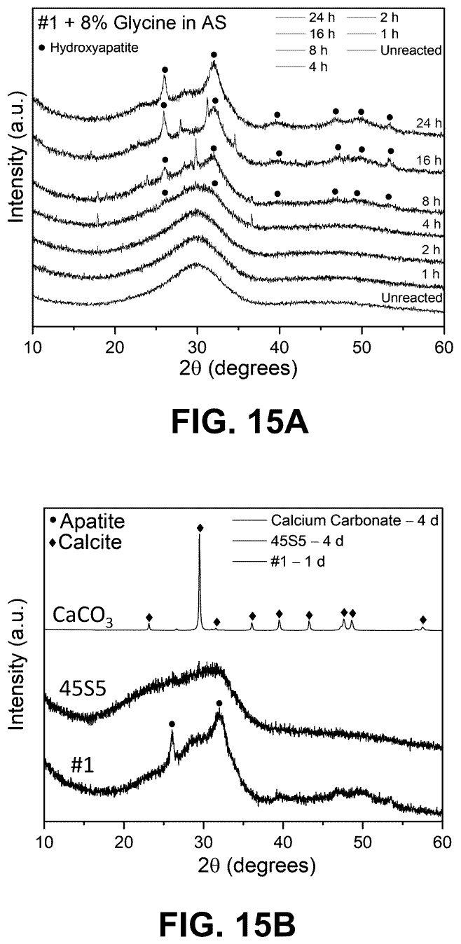

In this example, 5 wt. % Composition 1 bioactive glass from Table 3 was immersed in Table 5's artificial saliva composition additionally comprising 25 wt. % glycine at 37° C. for various time periods as shown in FIG. 15A, according to the details of the immersion experiments of Table 5. As shown in FIG. 15A, hydroxyapatite mineralization was observed within 4 hours after immersion., with continued mineralization occurring with greater immersion times.

As an additional experiment, 5 wt. % Composition 1 bioactive glass from Table 3 was immersed in Table 5's artificial saliva composition additionally comprising 8 wt. % glycine at 37° C. for 1 day according to the details of the immersion experiments of Table 5, with the XRD results shown in in FIG. 15B. This figure illustrates that apatite formation occurs within 1 day of immersion. For comparison, 5 wt. % 45S5 Bioglass® and 5 wt. % calcium carbonate were separately immersed in the same artificial saliva composition containing 8 wt. % glycine at 37° C. for 4 days, but no apatite formation was observed even after 4 days of immersion.

As shown in this example, including FIGS. 15A and 15B, Composition 1 bioactive glass demonstrates significantly superior hydroxyapatite mineralization (within 1 day in artificial saliva and 8% glycine) relative to 45S5 and calcium carbonate (significant mineralization is not observed even after 4 days in artificial saliva and 8% glycine). This unique rapid mineralization property of the combination of Composition 1 bioactive glass and the amino acid glycine enables their incorporation as active ingredients in a wide variety of applications that could benefit from such rapid mineralization rates, including but not limited to toothpaste, mouthwash, dental strips, and/or dental trays. Such rapid mineralization also enables less frequent use of such products.

### Example 6—Stability of Bioactive Glass

This example demonstrates the stability of the bioactive glasses disclosed herein as compared to 45S5 Bioglass®.

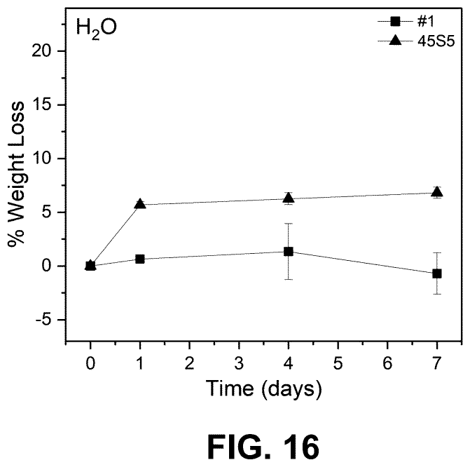

In this example, Composition 1 bioactive glass and 45S5 Bioglass®, both shown in Table 3, were separately immersed in deionized (DI) water at 37° C. for various time periods up to 7 days according to the details of the immersion experiments of Table 5. As shown in FIG. 16, Composition 1 bioactive glass showed improved water durability with respect to 45S5 Bioglass®, since virtually no weight loss was observed for Composition 1 even at 7 days of immersion. In contrast, 45S5 Bioglass® exhibited between about 5 to 7% weight loss over 1 to 7 days.

### Example 7—Human Enamel Treated with a Dentifrice Composition Containing Bioactive Glass and Amino Acid

This example demonstrates the ability to remineralize human enamel using a dentifrice composition (toothpaste) containing a bioactive glass and amino acid, as compared to a control dentifrice composition (toothpaste) without either the bioactive glass or amino acid. The intent of this example is to mimic typical brushing conditions (e.g., times, saliva, rinsing, etc.), except without performing any actual brushing.

The dentifrice composition in this example contains a toothpaste, namely, Sensodyne® Extra Whitening sold by Haleon, which lists the ingredients as follows: potassium nitrate, sodium fluoride, water, hydrated silica, sorbitol, glycerin, pentasodium triphosphate, PEG-8, flavor, titanium dioxide, sodium methyl cocoyl taurate, cocamidopropyl betaine, xanthan gum, sodium hydroxide, sodium saccharin, and sucralose. The human enamel discs were prepared as indicated in Example 4.

In this example, two toothpaste slurries were prepared: (1) Control Toothpaste Slurry and (2) Toothpaste Slurry A. In the Control Toothpaste Slurry, 2 parts by volume of the artificial saliva shown in Table 5 was mixed with 1 parts by volume of Sensodyne® Extra Whitening. Toothpaste Slurry A was prepared similarly, except the 1 part by volume portion was instead made up of a mixture of Sensodyne® Extra Whitening, 5 wt. % (relative to the mixture) Composition 1 bioactive glass shown in Table 3, and 8 wt. % (relative to the mixture) glycine.

Each slurry was then placed on different human enamel discs twice per day (morning and afternoon) for two minutes at ambient conditions, followed by washing off under flowing DI water. Following the afternoon treatment, the treated enamel was then soaked in artificial saliva at 37° C. for 12 hr, followed by sonicating the enamel in room temperature DI water for 10 minutes to completely remove any residual toothpaste slurry left on the enamel surface. The samples were then dried in a desiccator prior to SEM analysis.

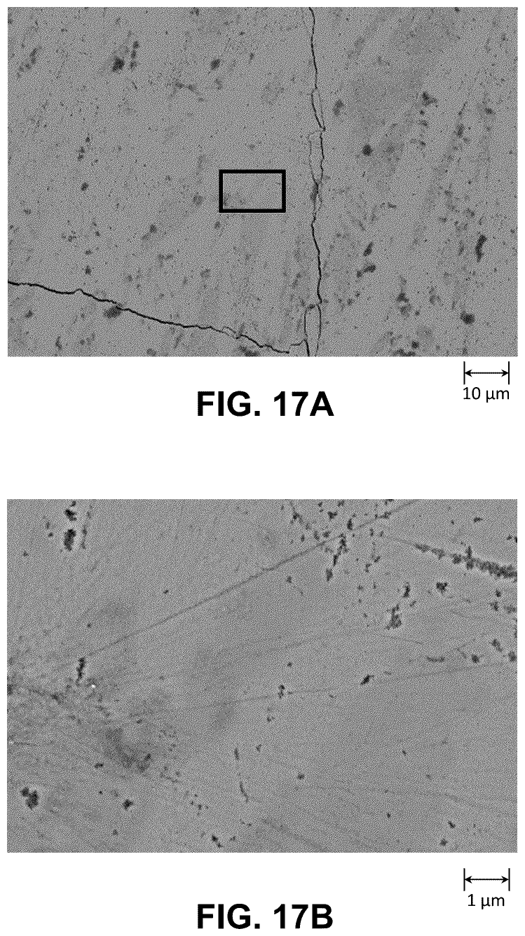

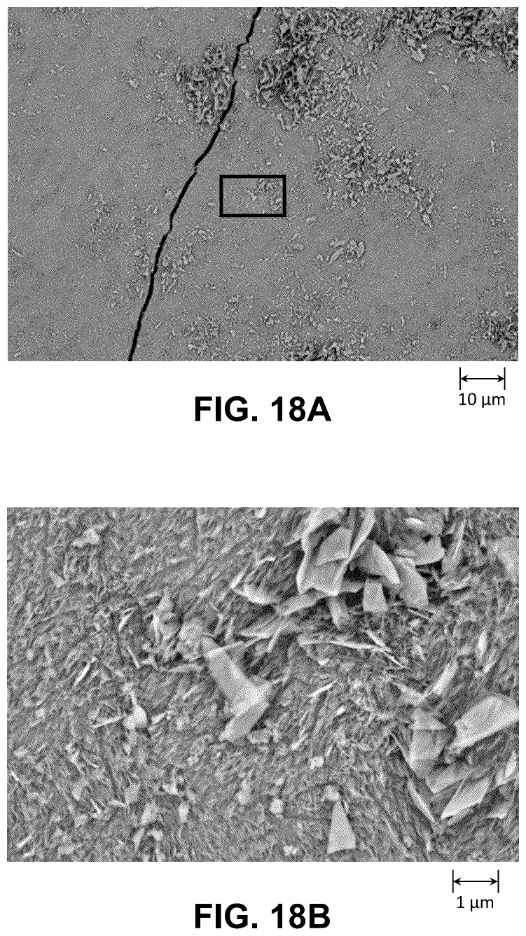

The results are depicted in the SEM images of FIGS. 17A, 17B, 18A, and 18B, which demonstrate the impact of the addition of bioactive glass and glycine in toothpaste on the remineralization of enamel Specifically, FIGS. 17A and 17B illustrate the surface morphology of enamel treated with the Control Toothpaste Slurry, showing noticeable voids and polishing scratches (FIG. 17B is a magnification of the boxed area of FIG. 17A). In contrast, FIGS. 18A and 18B illustrate the surface morphology of enamel treated with Toothpaste Slurry A (containing a bioactive glass and glycine), showing crystals on the enamel to cover the voids and scratches (FIG. 18B is a magnification of the boxed area of FIG. 18A).

The SEM parameters for FIGS. 17A-18B are as follows: FIG. 17A: CS Gemini 500; WD=6.9 mm; EHT=5.00 kV; Mag=1.00 K X; Signal A=BSD4 A; high vacuum; scale marks 10 microns; FIG. 17B: CS Gemini 500; WD=6.9 mm; EHT=5.00 kV; Mag=10.00 K X; Signal A=BSD4 A; high vacuum; scale marks 1 micron; FIG. 18A: CS Gemini 500; WD=6.6 mm; EHT=5.00 kV; Mag=1.00 K X; Signal A=BSD4 A; high vacuum; scale marks 10 microns; FIG. 17B: CS Gemini 500; WD=6.6 mm; EHT=5.00 kV; Mag=10.00 K X; Signal A=BSD4 A; high vacuum; scale marks 1 micron.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

References herein to the positions of elements (e.g., “top,” “bottom,” “above,” “below,” “first,” “second,” etc.) are merely used to describe the orientation of various elements in the FIGURES. It should be noted that the orientation of various elements may differ according to other exemplary embodiments, and that such variations are intended to be encompassed by the present disclosure. Moreover, these relational terms are used solely to distinguish one entity or action from another entity or action, without necessarily requiring or implying any actual such relationship or order between such entities or actions.

Modifications of the disclosure will occur to those skilled in the art and to those who make or use the disclosure. Therefore, it is understood that the embodiments shown in the drawings and described above are merely for illustrative purposes and not intended to limit the scope of the disclosure, which is defined by the following claims, as interpreted according to the principles of patent law, including the doctrine of equivalents.

It will be understood by one having ordinary skill in the art that construction of the described disclosure, and other components, is not limited to any specific material. Other exemplary embodiments of the disclosure disclosed herein may be formed from a wide variety of materials, unless described otherwise herein.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

As utilized herein, “optional,” “optionally,” or the like are intended to mean that the subsequently described event or circumstance can or cannot occur, and that the description includes instances where the event or circumstance occurs and instances where it does not occur. As used herein, the indefinite articles “a,” “an,” and the corresponding definite article “the” mean “at least one” or “one or more,” unless otherwise specified. It also is understood that the various features disclosed in the specification and the drawings can be used in any and all combinations.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

Unless otherwise specified, all compositions are expressed in terms of as-batched weight percent (wt. %). As will be understood by those having ordinary skill in the art, various melt constituents (e.g., silicon, alkali- or alkaline-based, boron, etc.) may be subject to different levels of volatilization (e.g., as a function of vapor pressure, melt time and/or melt temperature) during melting of the constituents. As such, the as-batched weight percent values used in relation to such constituents are intended to encompass values within ±0.5 wt. % of these constituents in final, as-melted articles. With the forgoing in mind, substantial compositional equivalence between final articles and as-batched compositions is expected.

It will be apparent to those skilled in the art that various modifications and variations can be made without departing from the spirit or scope of the claimed subject matter. Accordingly, the claimed subject matter is not to be restricted except in light of the attached claims and their equivalents.

What is claimed is:

1. A composition, comprising:
an amino acid; and
a bioactive glass, comprising:
15-55 wt. % SiO₂;
0.1-15 wt. % ZrO₂;
20-60 wt. % CaO; and
5-30 wt. % P₂O₅;
wherein, when the composition is subjected to artificial saliva of Table 1A or 1B at 37° C., a bioactive crystalline phase is formed within 7 days.

2. The composition of claim 1, wherein, when the composition is subjected to the artificial saliva of Table 1A or 1B at 37° C., the bioactive glass forms a bioactive crystalline phase within 1 day.

3. The composition of claim 1, wherein the bioactive crystalline phase comprises apatite, brushite, or a combination thereof.

4. The composition of claim 1, wherein the bioactive glass is present in an amount of >0-20 wt. %, based on total weight of the composition.

5. The composition of claim 1, wherein the bioactive glass comprises:
25-35 wt. % SiO₂;
35-50 wt. % CaO;
15-25 wt. % P₂O₅; and
3-12 wt. % ZrO₂, based on total weight of the bioactive glass;
based on total weight of the bioactive glass.

6. The composition of claim 1, wherein the bioactive glass comprises at least one of:
>0-25 wt. % MgO, based on total weight of the bioactive glass;
>0-5 wt. % F⁻, based on total weight of the bioactive glass;
0-5 wt. % Na₂O+K₂O+Li₂O, based on total weight of the bioactive glass;
CaO+P₂O₅ in an amount of 40-70 wt. %, based on total weight of the bioactive glass.

7. The composition of claim 1, wherein:
the amino acid is present in an amount of >0-25 wt. %, based on total weight of the composition.

8. The composition of claim 1, wherein the amino acid is glycine, glutamic acid, or a combination thereof.

9. The composition of claim 1, wherein the amino acid is arginine.

10. The composition of claim 1, wherein:
the composition is in a form of a toothpaste, mouthwash, gel, varnish, rinse, spray, ointment, salve, cream, bandage, polymer film, oral formulation, pill, capsule, transdermal formulation, or any combination thereof.

11. The composition of claim 1, wherein the composition is a dentifrice composition or a skin care composition.

12. The composition of claim 1, wherein the composition comprises:
water, glycerin, or both, and
one or more of a humectant, a fluoride ion source, a flavoring, a surfactant, a sweetener, a whitening agent, an abrasive, a thickening agent, a pH modifier, a preservative, or any combination thereof.

13. A method for making the composition of claim 1, comprising combining the amino acid, the bioactive glass, and optionally one or more of water, glycerin, a humectant, a fluoride ion source, a flavoring, a surfactant, a sweetener, a whitening agent, an abrasive, a thickening agent, a pH modifier, a preservative, or any combination thereof.

14. A method comprising applying to a tooth the composition of claim 1.

15. The method of claim 14, wherein the method remineralizes enamel of the tooth, treats caries of the tooth, treats dentin hypersensitivity of the tooth, or any combination thereof.

16. A method comprising applying to skin the composition of claim 1.

17. The method of claim 16, wherein the method treats or reduces the appearance of one or more skin defects, optionally wherein the one or more skin defects comprises wrinkles, blemishes, sagging skin, or any combination thereof.

18. A method for making the bioactive glass of the composition of claim 1, comprising:
combining each component of the bioactive glass to form a mixture,
heating the mixture to a temperature of 1500° C. or less to form a melted mixture, and
cooling the melted mixture to form a cooled mixture.

19. A dental strip or dental tray comprising the composition of claim 1.

20. A kit comprising a container comprising the composition of claim 1, in combination with a tray and/or flexible strips.
